SEQUENCE LISTING (74 sequences)

SEQ ID NO: 1 - protein, 672 aa, Artificial Sequence
  CAR
MALPVTALLLPLALLLHAARPEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - DNA, 2019 bp, Artificial Sequence
  CAR
atggccttaccagtgaccgccttgctcctgccgctggccttgctgctccacgccgccaggccggaagtgcagctggtggagtctgggggaggcttggtacagcctggcaggtccctgagactctcctgtgcagcctctggattcacctttaatgattatgccatgcactgggtccggcaagctccagggaagggcctggagtgggtctcaactattagttggaatagtggttccataggctatgcggactctgtgaagggccgattcaccatctccagagacaacgccaagaagtccctgtatctgcaaatgaacagtctgagagctgaggacacggccttgtattactgtgcaaaagatatacagtacggcaactactactacggtatggacgtctggggccaagggaccacggtcaccgtctcctcaggtggcggtggctcgggcggtggtgggtcgggtggcggcggatctgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaagagagcaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 3 - protein, 670 aa, Artificial Sequence
  CAR
MALPVTALLLPLALLLHAARPQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 2013 bp, Artificial Sequence
  CAR
atggccttaccagtgaccgccttgctcctgccgctggccttgctgctccacgccgccaggccgcaggtgcagttgcaacagcctggagctgagttggtgaagcctggtgcttctgtgaagatgtcttgtaaggcttctggatacacattcacttcttacaacatgcactgggtgaagcagactcctggtaggggtttggagtggatcggagctatctacccaggaaacggagacacatcttacaaccagaagttcaagggtaaggctacattgactgctgacaagtcttcatctactgcttacatgcaattgtcttctttgacatctgaggactctgcagtttactactgcgctaggtctacatactacggaggtgactggtacttcaacgtgtggggagcaggtaccacggtcactgtctctgcaggtggaggtggatctggaggaggaggatctggtggaggaggttctcaaattgttctctcccagtctccagcaatcctgtcagcttctcctggagagaaggtgactatgacttgcagggcttcttcatctgtttcttacatccactggttccagcagaagcctggttcttcacctaagccttggatctacgctacatctaacttggcatctggagtgcctgtgaggttctctggttctggttcaggtacttcttactctttgacaatctctagggtggaggctgaggacgctgctacttactactgccagcagtggacatctaaccctccaacattcggaggtggtactaagttggagatcaaggagagcaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 5 - protein, 672 aa, Artificial Sequence
  CAR
MALPVTALLLPLALLLHAARPEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 2019 bp, Artificial Sequence
  CAR
atggccttaccagtgaccgccttgctcctgccgctggccttgctgctccacgccgccaggccggaagtgcagctggtggagtctgggggaggcttggtacagcctggcaggtccctgagactctcctgtgcagcctctggattcacctttaatgattatgccatgcactgggtccggcaagctccagggaagggcctggagtgggtctcaactattagttggaatagtggttccataggctatgcggactctgtgaagggccgattcaccatctccagagacaacgccaagaagtccctgtatctgcaaatgaacagtctgagagctgaggacacggccttgtattactgtgcaaaagatatacagtacggcaactactactacggtatggacgtctggggccaagggaccacggtcaccgtctcctcaggtggcggtggctcgggcggtggtgggtcgggtggcggcggatctgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaagagagcaagtacggaccgccctgccccccttgccctgcccccgagttcgagggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccaaagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 7 - protein, 122 aa, Artificial Sequence
  Heavy chain variable region (VH)
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 8 - DNA, 366 bp, Artificial Sequence
  Heavy chain variable region (VH)
gaagtgcagctggtggagtctgggggaggcttggtacagcctggcaggtccctgagactctcctgtgcagcctctggattcacctttaatgattatgccatgcactgggtccggcaagctccagggaagggcctggagtgggtctcaactattagttggaatagtggttccataggctatgcggactctgtgaagggccgattcaccatctccagagacaacgccaagaagtccctgtatctgcaaatgaacagtctgagagctgaggacacggccttgtattactgtgcaaaagatatacagtacggcaactactactacggtatggacgtctggggccaagggaccacggtcaccgtctcctca

SEQ ID NO: 9 - protein, 121 aa, Artificial Sequence [sequence omitted]

SEQ ID NO: 10 - DNA, 363 bp, Artificial Sequence
  Heavy chain variable region (VH)
caggtgcagttgcaacagcctggagctgagttggtgaagcctggtgcttctgtgaagatgtcttgtaaggcttctggatacacattcacttcttacaacatgcactgggtgaagcagactcctggtaggggtttggagtggatcggagctatctacccaggaaacggagacacatcttacaaccagaagttcaagggtaaggctacattgactgctgacaagtcttcatctactgcttacatgcaattgtcttctttgacatctgaggactctgcagtttactactgcgctaggtctacatactacggaggtgactggtacttcaacgtgtggggagcaggtaccacggtcactgtctctgca

SEQ ID NO: 11 - protein, 107 aa, Artificial Sequence
  Light chain variable region (VL)
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 12 - DNA, 321 bp, Artificial Sequence
  Light chain variable region (VL)
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaa

SEQ ID NO: 13 - protein, 106 aa, Artificial Sequence [sequence omitted]

SEQ ID NO: 14 - DNA, 318 bp, Artificial Sequence
  Light chain variable region (VL)
caaattgttctctcccagtctccagcaatcctgtcagcttctcctggagagaaggtgactatgacttgcagggcttcttcatctgtttcttacatccactggttccagcagaagcctggttcttcacctaagccttggatctacgctacatctaacttggcatctggagtgcctgtgaggttctctggttctggttcaggtacttcttactctttgacaatctctagggtggaggctgaggacgctgctacttactactgccagcagtggacatctaaccctccaacattcggaggtggtactaagttggagatcaag

SEQ ID NO: 15 - protein, 15 aa, Artificial Sequence
  Linker Peptide
GGGGSGGGGSGGGGS

SEQ ID NO: 16 - DNA, 45 bp, Artificial Sequence
  Linker Peptide
ggtggcggtggctcgggcggtggtgggtcgggtggcggcggatct

SEQ ID NO: 17 - protein, 229 aa, Artificial Sequence
  Hinge region (H)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - DNA, 687 bp, Artificial Sequence
  Hinge region (H)
gagagcaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 19 - protein, 229 aa, Artificial Sequence
  Hinge region (H)
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - DNA, 687 bp, Artificial Sequence
  Hinge region (H)
gagagcaagtacggaccgccctgccccccttgccctgcccccgagttcgagggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccaaagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 21 - protein, 24 aa, Artificial Sequence
  Transmembrane domain (TM)
IYIWAPLAGTCGVLLLSLVITLYC

SEQ ID NO: 22 - DNA, 72 bp, Artificial Sequence
  Transmembrane domain (TM)
atctacatctgggcgcccttggccgggacttgtggggtccttctcctgtcactggttatcaccctttactgc

SEQ ID NO: 23 - protein, 42 aa, Artificial Sequence
  Co-stimulatory molecule (CS)
KRGRKKLLYIFKQPFMRPVQTTQEEDGCSCRFPEEEEGGCEL

SEQ ID NO: 24 - DNA, 126 bp, Artificial Sequence
  Co-stimulatory molecule (CS)
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 25 - protein, 113 aa, Artificial Sequence
  CD3zeta
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - DNA, 336 bp, Artificial Sequence
  CD3zeta
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - protein, 21 aa, Artificial Sequence
  Leader (L)
MALPVTALLLPLALLLHAARP

SEQ ID NO: 28 - DNA, 63 bp, Artificial Sequence
  Leader (L)
atggccttaccagtgaccgccttgctcctgccgctggccttgctgctccacgccgccaggccg

SEQ ID NO: 29 - protein, 677 aa, Artificial Sequence
  CAR
MALPVTALLLPLALLLHAARPQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSGGGGSGGGGSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 2034 bp, Artificial Sequence
  CAR
atggccttaccagtgaccgccttgctcctgccgctggccttgctgctccacgccgccaggccgcaggtgcaattggtgcagtctggcgctgaagttaagaagcctgggagttcagtgaaggtctcctgcaaggcttccggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcaggtggcggtggctcgggcggtggtgggtcgggtggcggcggatctgatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggctccgggtcaggcactgatttcacactgaaaatcagcagggtggaggctgaggatgttggagtttattactgcgctcagaatctagaacttccttacaccttcggcggagggaccaaggtggagatcaaacgtacggtggagagcaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 31 - protein, 716 aa, Artificial Sequence
  CAR
MALPVTALLLPLALLLHAARPEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 2151 bp, Artificial Sequence
  CAR
atggccttaccagtgaccgccttgctcctgccgctggccttgctgctccacgccgccaggccggaagtgcagctggtggagtctgggggaggcttggtacagcctggcaggtccctgagactctcctgtgcagcctctggattcacctttaatgattatgccatgcactgggtccggcaagctccagggaagggcctggagtgggtctcaactattagttggaatagtggttccataggctatgcggactctgtgaagggccgattcaccatctccagagacaacgccaagaagtccctgtatctgcaaatgaacagtctgagagctgaggacacggccttgtattactgtgcaaaagatatacagtacggcaactactactacggtatggacgtctggggccaagggaccacggtcaccgtctcctcaggtggcggtggctcgggcggtggtgggtcgggtggcggcggatctgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaagagagcaagtacggaccgccctgccccccttgccctgcccccgagttcgagggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccaaagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 33 - protein, 119 aa, Artificial Sequence
  Heavy chain variable region (VH)
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 34 - DNA, 357 bp, Artificial Sequence
  Heavy chain variable region (VH)
caggtgcaattggtgcagtctggcgctgaagttaagaagcctgggagttcagtgaaggtctcctgcaaggcttccggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctca

SEQ ID NO: 35 - protein, 115 aa, Artificial Sequence
  Light chain variable region (VL)
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 36 - DNA, 345 bp, Artificial Sequence
  Light chain variable region (VL)
gatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggctccgggtcaggcactgatttcacactgaaaatcagcagggtggaggctgaggatgttggagtttattactgcgctcagaatctagaacttccttacaccttcggcggagggaccaaggtggagatcaaacgtacggtg

SEQ ID NO: 37 - protein, 27 aa, Artificial Sequence
  Transmembrane domain (TM)
FWVLVVVGGVLACYSLLVTVAFIIFWV

SEQ ID NO: 38 - DNA, 81 bp, Artificial Sequence
  Transmembrane domain (TM)
ttttgggtgctggtggtggttggtggagtcctggcttgctatagcttgctagtaacagtggcctttattattttctgggtg

SEQ ID NO: 39 - protein, 41 aa, Artificial Sequence
  Co-stimulatory molecule (CS)
RSKRSRLLHSDYMNMTPRRPGPTRKHYQPYAPPRDFAAYRS

SEQ ID NO: 40 - DNA, 123 bp, Artificial Sequence
  Co-stimulatory molecule (CS)
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 41 - protein, 6 aa, Artificial Sequence
  synthetic
NDYAMH

SEQ ID NO: 42 - protein, 17 aa, Artificial Sequence
  synthetic
TISWNSGSIGYADSVKG

SEQ ID NO: 43 - protein, 13 aa, Artificial Sequence
  synthetic
DIQYGNYYYGMDV

SEQ ID NO: 44 - protein, 11 aa, Artificial Sequence
  synthetic
RASQSVSSYLA

SEQ ID NO: 45 - protein, 7 aa, Artificial Sequence
  synthetic
DASNRAT

SEQ ID NO: 46 - protein, 9 aa, Artificial Sequence
  synthetic
QQRSNWPIT

SEQ ID NO: 47 - protein, 30 aa, Artificial Sequence
  synthetic
QVQLQQPGAELVKPGASVKMSCKASGYTFT

SEQ ID NO: 48 - protein, 5 aa, Artificial Sequence
  synthetic
SYNMH

SEQ ID NO: 49 - protein, 14 aa, Artificial Sequence
  synthetic
WVKQTPGRGLEWIG

SEQ ID NO: 50 - protein, 17 aa, Artificial Sequence
  synthetic
AIYPGNGDTSYNQKFKG

SEQ ID NO: 51 - protein, 32 aa, Artificial Sequence
  synthetic
KATLTADKSSSTAYMQLSSLTSEDSAVYYCAR

SEQ ID NO: 52 - protein, 12 aa, Artificial Sequence
  synthetic
STYYGGDWYFNV

SEQ ID NO: 53 - protein, 11 aa, Artificial Sequence
  synthetic
WGAGTTVTVSA

SEQ ID NO: 54 - protein, 23 aa, Artificial Sequence
  synthetic
QIVLSQSPAILSASPGEKVTMTC

SEQ ID NO: 55 - protein, 10 aa, Artificial Sequence
  synthetic
RASSSVSYIH

SEQ ID NO: 56 - protein, 15 aa, Artificial Sequence
  synthetic
WFQQKPGSSPKPWIY

SEQ ID NO: 57 - protein, 7 aa, Artificial Sequence
  synthetic
ATSNLAS

SEQ ID NO: 58 - protein, 32 aa, Artificial Sequence
  synthetic
GVPVRFSGSGSGTSYSLTISRVEAEDAATYYC

SEQ ID NO: 59 - protein, 9 aa, Artificial Sequence
  synthetic
QQWTSNPPT

SEQ ID NO: 60 - protein, 10 aa, Artificial Sequence
  synthetic
FGGGTKLEIK

SEQ ID NO: 61 - protein, 30 aa, Artificial Sequence
  synthetic
QVQLVQSGAEVKKPGSSVKVSCKASGYAFS

SEQ ID NO: 62 - protein, 5 aa, Artificial Sequence
  synthetic
YSWIN

SEQ ID NO: 63 - protein, 14 aa, Artificial Sequence
  synthetic
WVRQAPGQGLEWMG

SEQ ID NO: 64 - protein, 17 aa, Artificial Sequence
  synthetic
RIFPGDGDTDYNGKFKG

SEQ ID NO: 65 - protein, 32 aa, Artificial Sequence
  synthetic
RVTITADKSTSTAYMELSSLRSEDTAVYYCAR

SEQ ID NO: 66 - protein, 10 aa, Artificial Sequence
  synthetic
NVFDGYWLVY

SEQ ID NO: 67 - protein, 11 aa, Artificial Sequence
  synthetic
WGQGTLVTVSS

SEQ ID NO: 68 - protein, 23 aa, Artificial Sequence
  synthetic
DIVMTQTPLSLPVTPGEPASISC

SEQ ID NO: 69 - protein, 16 aa, Artificial Sequence
  synthetic
RSSKSLLHSNGITYLY

SEQ ID NO: 70 - protein, 15 aa, Artificial Sequence
  synthetic
WYLQKPGQSPQLLIY

SEQ ID NO: 71 - protein, 7 aa, Artificial Sequence
  synthetic
QMSNLVS

SEQ ID NO: 72 - protein, 32 aa, Artificial Sequence
  synthetic
GVPDRFSGSGSGTDFTLKISRVEAEDVGVYYC

SEQ ID NO: 73 - protein, 9 aa, Artificial Sequence
  synthetic
AQNLELPYT

SEQ ID NO: 74 - protein, 13 aa, Artificial Sequence
  synthetic
FGGGTKVEIKRTV

(19) United States
(12) Patent Application Publication
(10) Pub. No.: US 2024/0115605 A1
(43) Pub. Date: Apr. 11, 2024

(54) CHIMERIC ANTIGEN RECEPTORS TARGETING CD20

(71) Applicant: CELLULAR BIOMEDICINE GROUP, INC., Rockville, MD (US)

(72) Inventors: Yihong YAO, Rockville, MD (US); Jiaqi HUANG, Rockville, MD (US); Xin YAO, Rockville, MD (US)

(21) Appl. No.: 18/263,050

(22) PCT Filed: Jan. 26, 2022
(86) PCT No.: PCT/US22/13875

§ 371 Date: Jul. 26, 2023

Related U.S. Application Data
(63) Continuation-in-part of application No. 17/352,915, filed on Jun. 21, 2021, now Pat. No. 11,472,858.
(60) Provisional application No. 63/154,040, filed on Feb. 26, 2021.
(60) Provisional application No. 63/142,216, filed on Jan. 27, 2021.

(51) Int. Cl. A61K 35/17; A61K 39/00; C07K 16/28

(52) U.S. Cl. CPC A61K 35/17; A61K 39/4611; A61K 39/4631; A61K 39/464424; C07K 16/2887; A61K 2239/15; A61K 2239/17; A61K 2239/21; C07K 2317/53; C07K 2317/565; C07K 2317/622

(57) ABSTRACT

Chimeric antigen receptors targeting CD20 and preparation methods thereof are provided. The antigen binding region of the chimeric antigen receptor may include a heavy chain variable region shown in SEQ ID NOs: 7, 9 or 33 and a light chain variable region shown in SEQ ID NOs: 11, 13 or 35.

### CROSS REFERENCE TO RELATED APPLICATIONS

The present application claims priority to U.S. Provisional Application Nos. 63/142,216 (filed on Jan. 27, 2021), and 63/154,040 (filed on Feb. 26, 2021), and U.S. application Ser. No. 17/352,915 (filed on Jun. 21, 2021), each of which is hereby incorporated by reference in its entirety.

### SEQUENCE LISTING

This application contains a Sequence Listing which has been filed electronically in ASCII format and is hereby incorporated by reference in its entirety. Said ASCII copy, created on Jan. 26, 2022, is named 11299-008883-WO1_ST25.txt and is 73 KB in size.

### TECHNICAL FIELD

The present disclosure provides chimeric antigen receptors targeting the CD20 antigen, and a preparation method for modified T cells (CAR-T cells) and activity identification thereof. The present disclosure provides chimeric antigen receptors for treating CD20-positive diseases such as B cell lymphoma.

### BACKGROUND

Malignant tumors of the blood system account for about 10% of human malignant tumors, and 95% of malignant tumors of the blood system are derived from B lymphocytes. Traditional chemotherapy and radiotherapy play an important role in the treatment of malignant tumors of the blood system. Some patients have seen significant effects, but most of them are difficult to cure. New and effective treatments are urgently needed.

Adoptive T cell therapy has shown its powerful efficacy and bright prospect in the clinical treatment of malignant tumors. Among them, multiple centers independently using chimeric antigen receptor (CAR)-modified T cells to target recurrent, refractory malignant tumors of CD19-expressed B cell have achieved unprecedented success. In particular, in a clinical trial carried out at the School of Medicine, University of Pennsylvania using CART19 in the treatment of recurrent, refractory acute B-cell lymphoma (R/R B-ALL), up to 94% of patients achieved complete remission. Although the initial response rate of this clinical trial was high, nearly 40% of patients who achieved complete response after 1 month of treatment, had a relapse, and more than 60% of patients with relapse had CD19-negative tumor cells escape. Therefore, there is an urgent need to identify CARs that target B cell lymphoma-associated antigens other than CD19 to treat patients with malignant lymphoma.

CD20 is a glycosylated protein and is the first identified B cell membrane marker. CD20 is also known as B1, and encoded by the MS4A gene. CD20 molecule has four transmembrane hydrophobic regions, and its N-terminal and C-terminal are located on the cytoplasmic side, thereby forming two closed loops outside the cell, which are respectively called big loop and small loop. CD20 is specifically expressed in more than 95% of normal and cancerous B cells. These cells are in the pre-B cell stage and subsequent developmental stages, and CD20 stops expression until the cells differentiated into plasma cells. Therefore, CD20 is an ideal target for immunotherapy of B cell malignancies.

Rituximab (MabThera®, Rituxan®) is the first generation of chimeric monoclonal antibody targeting CD20 which was firstly approved by the US FDA and the European EMA for treating indolent lymphoma. Rituximab recognizes and binds to the big loop structure of the extracellular domain of CD20, and it kills tumor cells by ADCC-mediated killing effect. However, Rituximab alone shows limited activity and short duration of response, but its combination with chemotherapy can significantly enhance the efficacy of chemotherapy. Rituximab is used for the treatment of lymphoma, and half of the patients have a complete response (CR) or a partial response (PR).

Ofatumumab (Arzerra®) is the first completely humanized CD20 therapeutic antibody. Unlike Rituximab, the epitope recognized by Ofatumumab contains parts of the big loop and the small loop of CD20. At the same time, the tumor killing method of Ofatumumab is mainly through the complement-dependent pathway, followed by ADCC-dependent tumor killing effect.

Obinutuzumab (Gazyvaro®, Gazyva®) is a humanized type II CD20 antibody that reduces fucosylation levels and optimizes FcγRIIIa affinity. Obinutuzumab recognizes and binds to the big loop of the extracellular molecule of CD20, and mediates the killing effect on tumor mainly through the ADCC effect. At the same time, the binding of Obinutuzumab to CD20 molecule also has the effect of inducing apoptosis of tumor cells. As for the NHL that does not respond to Rituximab treatment, Obinutuzumab is combined with bendamustine, a nitrogen mustard drug. The phase III clinical trial found that the duration with no deterioration of combination therapy of Obinutuzumab and bendamustine was twice as long as that of bendamustine therapy alone (the former is 29 months and the latter is 14 months). Obinutuzumab has an overall response rate (ORR, including CR and PR) of 77.3%, and Rituximab is 65.7%.

Compared with therapeutic antibodies, cellular immunotherapy is an emerging and highly effective tumor treatment model, and is a new type of autoimmunolgy treatment for cancer. It is a method for in vitro culture and amplification of immune cells collected from a patient using biotechnology and biological agents, and then the cells are transfused back to the patient to stimulate and enhance the body's autoimmune function, thereby achieving the purpose of treating tumors. The skilled in the art have been working to develop new cellular immunotherapy to increase its efficiency and reduce its side effects. Although many therapeutic antibodies as described above have been developed in these years, their clinical therapeutic effects have not reached the same level of therapeutic effects as CART19. Therefore, the development of CART therapy targeting CD20 has great market value and social significance.

### SUMMARY

The present disclosure provides for a chimeric antigen receptor (CAR), comprising: an anti-CD20 antigen-binding region which comprises a heavy chain variable region (V_{H}) and a light chain variable region (V_{L}), V_{H} comprising three CDRs, HCDR1, HCDR2 and HCDR3, V_{L} comprising three complementarity determining regions (CDRs), LCDR1, LCDR2 and LCDR3.

In certain embodiments, HCDR1, HCDR2 and HCDR3 may have amino acid sequences about 80% to about 100% identical to the amino acid sequences set forth in SEQ ID NO: 41, SEQ ID NO: 42, SEQ ID NO: 43, respectively. LCDR1, LCDR2 and LCDR3 may have amino acid sequences about 80% to about 100% identical to the amino acid sequences set forth in SEQ ID NO: 44, SEQ ID NO: 45, SEQ ID NO: 46, respectively.

In certain embodiments, HCDR1, HCDR2 and HCDR3 may have amino acid sequences about 80% to about 100% identical to the amino acid sequences set forth in SEQ ID NO: 48, SEQ ID NO: 50, SEQ ID NO: 52, respectively. LCDR1, LCDR2 and LCDR3 may have amino acid sequences about 80% to about 100% identical to the amino acid sequences set forth in SEQ ID NO: 55, SEQ ID NO: 57, SEQ ID NO: 59, respectively.

In certain embodiments, HCDR1, HCDR2 and HCDR3 may have amino acid sequences about 80% to about 100% identical to the amino acid sequences set forth in SEQ ID NO: 62, SEQ ID NO: 64, SEQ ID NO: 66, respectively. LCDR1, LCDR2 and LCDR3 may have amino acid sequences about 80% to about 100% identical to the amino acid sequences set forth in SEQ ID NO: 69, SEQ ID NO: 71, SEQ ID NO: 73, respectively.

In certain embodiments, V_{H} is located at the N-terminus of V_{L}. In certain embodiments, V_{L} is located at the N-terminus of V_{H}.

In certain embodiments, V_{H} and V_{L} have amino acid sequences about 80% to about 100% identical to amino acid sequences set forth in (a) SEQ ID NO: 7 and SEQ ID NO: 11, respectively; (b) SEQ ID NO: 9 and SEQ ID NO: 13, respectively; or (c) SEQ ID NO: 33 and SEQ ID NO: 35, respectively.

In certain embodiments, the anti-CD20 antigen-binding region is a single-chain variable fragment (scFv) that specifically binds CD20.

The CAR may further comprise one or more of the following: (a) a signal peptide, (b) a hinge region, (c) a transmembrane domain, (d) a co-stimulatory region, and (e) a cytoplasmic signaling domain.

In certain embodiments, the co-stimulatory region comprises a co-stimulatory region of 4-1BB (CD137), CD28, or combinations thereof. In certain embodiments, the co-stimulatory region comprises an amino acid sequence about 80% to about 100% identical to the amino acid sequence set forth in SEQ ID NO: 23, or SEQ ID NO: 39.

In certain embodiments, the cytoplasmic signaling domain comprises a cytoplasmic signaling domain of CD3ζ. In certain embodiments, the cytoplasmic signaling domain comprises an amino acid sequence about 80% to about 100% identical to the amino acid sequence set forth in SEQ ID NO: 25.

In certain embodiments, the hinge region comprises a hinge region of CD8, CD28, CD137, IG4, or combinations thereof. In certain embodiments, the hinge region comprises an amino acid sequence about 80% to about 100% identical to the amino acid sequence set forth in SEQ ID NO: 17, or SEQ ID NO: 19.

In certain embodiments, the transmembrane domain comprises a transmembrane domain of CD8, CD28, or combinations thereof. In certain embodiments, the transmembrane domain comprises an amino acid sequence about 80% to about 100% identical to the amino acid sequence set forth in SEQ ID NO: 21.

In certain embodiments, the CAR comprises an amino acid sequence about 80% to about 100% identical to the amino acid sequence set forth in SEQ ID NO: 1, SEQ ID NO: 3, SEQ ID NO: 5, SEQ ID NO: 29, or SEQ ID NO: 31. In one embodiment, the CAR comprises an amino acid sequence about 80% to about 100% identical to the amino acid sequence set forth in SEQ ID NO: 5.

The present disclosure provides for an immune cell expressing or comprising the CAR. The immune cell may be a T cell or a natural killer (NK) cell.

The present disclosure also provides for a nucleic acid encoding the CAR, or a vector comprising the nucleic acid.

The present disclosure provides for a pharmaceutical composition comprising the immune cell, the nucleic acid, the vector, or the CAR.

Also encompassed by the present disclosure is a method of treating cancer, the method comprising administering the immune cell to a subject in need thereof.

The cancer may be a hematologic cancer. The cancer may be a B-cell malignancy. The B-cell malignancy may be acute lymphocytic leukemia (ALL), chronic lymphocytic leukemia (CLL), B-cell acute lymphoblastic leukemia (B-ALL), B-cell leukemia, or B cell lymphoma.

The cancer may be Hodgkin's lymphoma, non-Hodgkin's lymphoma, leukemia, and/or multiple myeloma (MM).

The immune cell may be administered by infusion, injection, transfusion, implantation, and/or transplantation. The immune cell may be administered intravenously, subcutaneously, intradermally, intranodally, intratumorally, intramedullary, intramuscularly, or intraperitoneally. The immune cell may be administered via intravenous infusion.

The immune cell may be allogeneic or autologous.

The subject may be a human.

The present disclosure provides for a method for treating cancer. The method may comprise administering the immune cell to a subject in need thereof. The chimeric antigen receptor (CAR) may generate an area under the curve (AUC) ranging from about 1.0e+05 copies/μg genomic DNA (copies/gDNA) to about 1.1e+07 copies/gDNA, from about 2.0e+06 copies/μg genomic DNA (copies/gDNA) to about 1.1e+07 copies/gDNA, from about 1.0e+05 copies/μg genomic DNA (copies/gDNA) to about 4.0e+06 copies/gDNA, from about 1.0e+06 copies/μg genomic DNA (copies/gDNA) to about 1.0e+07 copies/gDNA, from about 5.0e+05 copies/μg genomic DNA (copies/gDNA) to about 1.3e+07 copies/gDNA, from about 5.0e+06 copies/μg genomic DNA (copies/gDNA) to about 1.0e+07 copies/gDNA, from about 5.0e+06 copies/μg genomic DNA (copies/gDNA) to about 1.3e+07 copies/gDNA, or from about 7.0e+06 copies/μg genomic DNA (copies/gDNA) to about 1.0e+07 copies/gDNA, in the blood of the subject in about 28 days after administration.

Also encompassed by the present disclosure is a method for treating cancer, the method comprising administering the immune cell to a subject in need thereof. The chimeric antigen receptor (CAR) may generate a maximum plasma concentration (Cₘₐₓ) ranging from about 1.0e+04 copies/μg genomic DNA (copies/gDNA) to about 1.1e+06 copies/gDNA, from about 1.0e+04 copies/μg genomic DNA (copies/gDNA) to about 3.0e+05 copies/gDNA, from about 2.0e+05 copies/μg genomic DNA (copies/gDNA) to about 1.1e+06 copies/gDNA, from about 5×10⁴ copies/μg genomic DNA (copies/gDNA) to about 1.3×10⁶ copies/gDNA, from about 5×10⁵ copies/μg genomic DNA (copies/gDNA) to about 1.3×10⁶ copies/gDNA, or from about 7.5×10⁵ copies/μg genomic DNA (copies/gDNA) to about 1×10⁶ copies/gDNA, in the blood of the subject. The CAR may have a Tₘₐₓ ranging from about 10 days to about 25 days, from about 10 days to about 20 days, from about 12 days to about 15 days, from about 12 days to about 25 days, from about 14 days to about 20 days, or from about 6 days to about 22 days.

In certain embodiments, the anti-CD20 antigen-binding region includes a heavy chain variable region (V_{H}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 7, SEQ ID NO: 9, or SEQ ID NO: 33.

In certain embodiments, the anti-CD20 antigen-binding region includes a light chain variable region (V_{L}) comprising an amino acid sequence at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100% identical to the amino acid sequence set forth in SEQ ID NO: 11, SEQ ID NO: 13, or SEQ ID NO: 35.

A heavy chain variable region of the anti-CD20 antigen-binding region can comprise one, two, or three complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the CDRs of a heavy chain variable region of the Ofatumumab antibody (CDR1, CDR2 and CDR3 as set forth in SEQ ID NO: 41, SEQ ID NO: 42, SEQ ID NO: 43, respectively), or the CDRs of a heavy chain variable region of the Rituximab antibody (CDR1, CDR2 and CDR3 as set forth in SEQ ID NO: 48, SEQ ID NO: 50, SEQ ID NO: 52, respectively), or the CDRs of a heavy chain variable region of the Obinutuzumab antibody (CDR1, CDR2 and CDR3 as set forth in SEQ ID NO: 62, SEQ ID NO: 64, SEQ ID NO: 66, respectively).

A light chain variable region of the anti-CD20 antigen-binding region can comprise one, two, or three complementarity determining regions (CDRs) that are at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the CDRs of a light chain variable region of the Ofatumumab antibody (CDR1, CDR2 and CDR3 as set forth in SEQ ID NO: 44, SEQ ID NO: 45, SEQ ID NO: 46, respectively), or the CDRs of a light chain variable region of the Rituximab antibody (CDR1, CDR2 and CDR3 as set forth in SEQ ID NO: 55, SEQ ID NO: 57, SEQ ID NO: 59, respectively), or the CDRs of a light chain variable region of the Obinutuzumab antibody (CDR1, CDR2 and CDR3 as set forth in SEQ ID NO: 69, SEQ ID NO: 71, SEQ ID NO: 73, respectively).

In certain embodiments, a heavy chain variable region of the anti-CD20 antigen-binding region includes three CDRs that are identical (e.g., 80%-100% identical) to the CDRs of a heavy chain variable region of the Ofatumumab antibody (CDR1, CDR2 and CDR3 as set forth in SEQ ID NO: 41, SEQ ID NO: 42, SEQ ID NO: 43, respectively), and a light chain variable region of the anti-CD20 antigen-binding region includes three CDRs that are identical (e.g., 80%-100% identical) to the CDRs of a light chain variable region of the Ofatumumab antibody (CDR1, CDR2 and CDR3 as set forth in SEQ ID NO: 44, SEQ ID NO: 45, SEQ ID NO: 46, respectively).

In certain embodiments, a heavy chain variable region of the anti-CD20 antigen-binding region includes three CDRs that are identical (e.g., 80%-100% identical) to the CDRs of a heavy chain variable region of the Rituximab antibody (CDR1, CDR2 and CDR3 as set forth in SEQ ID NO: 48, SEQ ID NO: 50, SEQ ID NO: 52, respectively), and a light chain variable region of the anti-CD20 antigen-binding region includes three CDRs that are identical (e.g., 80%-100% identical) to the CDRs of a light chain variable region of the Rituximab antibody (CDR1, CDR2 and CDR3 as set forth in SEQ ID NO: 55, SEQ ID NO: 57, SEQ ID NO: 59, respectively).

In certain embodiments, a heavy chain variable region of the anti-CD20 antigen-binding region includes three CDRs that are identical (e.g., 80%-100% identical) to the CDRs of a heavy chain variable region of the Obinutuzumab antibody (CDR1, CDR2 and CDR3 as set forth in SEQ ID NO: 62, SEQ ID NO: 64, SEQ ID NO: 66, respectively), and a light chain variable region of the anti-CD20 antigen-binding region includes three CDRs that are identical (e.g., 80%-100% identical) to the CDRs of a light chain variable region of the Obinutuzumab antibody (CDR1, CDR2 and CDR3 as set forth in SEQ ID NO: 69, SEQ ID NO: 71, SEQ ID NO: 73, respectively).

In certain embodiments, the CAR may comprise an amino acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 1, SEQ ID NO: 3, SEQ ID NO: 5, SEQ ID NO: 29, or SEQ ID NO: 31.

In certain embodiments, the nucleic acid encoding the CAR may comprise a nucleic acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the nucleic acid sequence set forth in SEQ ID NO: 2, SEQ ID NO: 4, SEQ ID NO: 6, SEQ ID NO: 30, or SEQ ID NO: 32.

In certain embodiments, the CAR may generate an area under the curve (AUC) ranging from about 1.0e+05 copies/μg genomic DNA (copies/gDNA) to about 1.1e+07 copies/gDNA, from about 2.0e+06 copies/μg genomic DNA (copies/gDNA) to about 1.1e+07 copies/gDNA, from about 1.0e+05 copies/μg genomic DNA (copies/gDNA) to about 4.0e+06 copies/gDNA, from about 1.0e+06 copies/μg genomic DNA (copies/gDNA) to about 1.0e+07 copies/gDNA, from about 0.5e+06 copies/μg genomic DNA (copies/gDNA) to about 2e+07 copies/gDNA, from about 5.0e+05 copies/μg genomic DNA (copies/gDNA) to about 1.3e+07 copies/gDNA, from about 5.0e+05 copies/μg genomic DNA (copies/gDNA) to about 2e+07 copies/gDNA, from about 5.0e+05 copies/μg genomic DNA (copies/gDNA) to about 1.5e+07 copies/gDNA, from about 5.0e+06 copies/μg genomic DNA (copies/gDNA) to about 1.0e+07 copies/gDNA, from about 5.0e+06 copies/μg genomic DNA (copies/gDNA) to about 1.3e+07 copies/gDNA, from about 7.0e+06 copies/μg genomic DNA (copies/gDNA) to about 1.0e+07 copies/gDNA, from about 8.0e+06 copies/μg genomic DNA (copies/gDNA) to about 1.0e+07 copies/gDNA, from about 0.5e+06 copies/μg genomic DNA (copies/gDNA) to about 4e+06 copies/gDNA, from about 0.5e+06 copies/μg genomic DNA (copies/gDNA) to about 3.5e+06 copies/gDNA, from about 1e+06 copies/μg genomic DNA (copies/gDNA) to about 3.5e+06 copies/gDNA, from about 1.2e+06 copies/μg genomic DNA (copies/gDNA) to about 3.2e+06 copies/gDNA, from about 0.8e+06 copies/μg genomic DNA (copies/gDNA) to about 3.2e+06 copies/gDNA, from about 1.6e+06 copies/μg genomic DNA (copies/gDNA) to about 3.2e+06 copies/gDNA, from about 1e+06 copies/μg genomic DNA (copies/gDNA) to about 2e+06 copies/gDNA, from about 0.6e+06 copies/μg genomic DNA (copies/gDNA) to about 1.8e+06 copies/gDNA, from about 3e+06 copies/μg genomic DNA (copies/gDNA) to about 3.2e+06 copies/gDNA, from about 0.5e+06 copies/μg genomic DNA (copies/gDNA) to about 1.7e+06 copies/gDNA, from about 2e+06 copies/μg genomic DNA (copies/gDNA) to about 3.2e+06 copies/gDNA, from about 1.5e+06 copies/μg genomic DNA (copies/gDNA) to about 2e+06 copies/gDNA, or from about 1e+06 copies/μg genomic DNA (copies/gDNA) to about 3.2e+06 copies/gDNA, in the blood of the subject in about 28 days after administration of the CAR to the subject. The AUC may be a median AUC.

In certain embodiments, the CAR generates a maximum plasma concentration (Cₘₐₓ) ranging from about 1.0e+04 copies/μg genomic DNA (copies/gDNA) to about 1.1e+06 copies/gDNA, from about 1.0e+04 copies/μg genomic DNA (copies/gDNA) to about 3.0e+05 copies/gDNA, from about 2.0e+05 copies/μg genomic DNA (copies/gDNA) to about 1.1e+06 copies/gDNA, from about 5×10⁴ copies/μg genomic DNA (copies/gDNA) to about 1.3×10⁶ copies/gDNA, from about 5×10⁴ copies/μg genomic DNA (copies/gDNA) to about 1.5×10⁶ copies/gDNA, from about 5×10⁵ copies/μg genomic DNA (copies/gDNA) to about 1.3×10⁶ copies/gDNA, from about 7.5×10⁵ copies/μg genomic DNA (copies/gDNA) to about 1×10⁶ copies/gDNA, from about 7×10⁵ copies/μg genomic DNA (copies/gDNA) to about 1×10⁶ copies/gDNA, from about 8×10⁵ copies/μg genomic DNA (copies/gDNA) to about 1×10⁶ copies/gDNA, from about 7.5'10⁵ copies/μg genomic DNA (copies/gDNA) to about 1.5×10⁶ copies/gDNA, from about 7×10⁵ copies/μg genomic DNA (copies/gDNA) to about 1.5×10⁶ copies/gDNA, from about 8×10⁵ copies/μg genomic DNA (copies/gDNA) to about 1.5×10⁶ copies/gDNA, from about 0.8e+05 copies/μg genomic DNA (copies/gDNA) to about 3.5e+05 copies/gDNA, from about 1e+05 copies/μg genomic DNA (copies/gDNA) to about 3.5e+05 copies/gDNA, from about 1e+05 copies/μg genomic DNA (copies/gDNA) to about 1.6e+05 copies/gDNA, from about 1e+05 copies/μg genomic DNA (copies/gDNA) to about 3.3e+05 copies/gDNA, from about 0.8e+05 copies/μg genomic DNA (copies/gDNA) to about 1.5e+05 copies/gDNA, from about 0.8e+05 copies/μg genomic DNA (copies/gDNA) to about 2e+05 copies/gDNA, from about 1e +05 copies/μg genomic DNA (copies/gDNA) to about 2e+05 copies/gDNA, from about 2e+05 copies/μg genomic DNA (copies/gDNA) to about 3e+05 copies/gDNA, from about 2e+05 copies/μg genomic DNA (copies/gDNA) to about 3.5e+05 copies/gDNA, from about 2e+05 copies/μg genomic DNA (copies/gDNA) to about 2.5e+05 copies/gDNA, or from about 1e+05 copies/μg genomic DNA (copies/gDNA) to about 3e+05 copies/gDNA, in the blood of the subject after administration of the CAR to the subject. The Cₘₐₓ may be a median Cₘₐₓ.

In certain embodiments, the CAR has a Tₘₐₓ (the time it takes the CAR to reach Cₘₐₓ) ranging from about 10 days to about 25 days, from about 10 days to about 20 days, from about 12 days to about 15 days, from about 12 days to about 25 days, from about 14 days to about 20 days, from about 6 days to about 22 days, from about 3 days to about 20 days, from about 4 days to about 18 days, from about 5 days to about 17 days, from about 6 days to about 16 days, from about 7 days to about 15 days, from about 9 days to about 15 days, from about 10 days to about 15 days, from about 10 days to about 14 days, from about 8 days to about 12 days, from about 6 days to about 14 days, from about 12 days to about 14 days, from about 8 days to about 11 days, from about 8 days to about 15 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, or from about 10 days to about 14 days. The Tₘₐₓ may be a median Tₘₐₓ.

In certain embodiments, the CAR has a Tₗₐₛₜ (the time corresponding to the last quantifiable CAR level) ranging from about 10 days to about 200 days, from about 10 days to about 100 days, from about 10 days to about 90 days, from about 50 days to about 80 days, from about 70 days to about 90 days, from about 30 days to about 90 days, from about 30 days to about 80 days, from about 30 days to about 200 days, from about 50 days to about 150 days, from about 50 days to about 100 days, from about 60 days to about 80 days, from about 60 days to about 150 days, from about 80 days to about 150 days, from about 50 days to about 200 days, from about 50 days to about 60 days, from about 50 days to about 80 days, from about 50 days to about 100 days, from about 60 days to about 100 days, from about 80 days to about 100 days, from about 60 days to about 200 days, from about 80 days to about 200 days, from about 50 days to about 140 days, from about 60 days to about 140 days, or from about 80 days to about 140 days. The Tₗₐₛₜ may be a median Tₗₐₛₜ.

In view of the differences in affinity and killing mechanisms of the therapeutic antibodies targeting CD20, we constructed a series of chimeric antigen receptors targeting CD20 using the antigen-binding regions of different antibodies, and completed the identification of anti-tumor activity and differential comparison of these chimeric antigen receptor T cells in vitro. The disclosure provides new and effective methods and preparations for clinical application of CAR-T in the treatment of CD20-positive leukemia and lymphoma.

It is an object of the present disclosure to provide chimeric antigen receptors targeting CD20, a preparation method and application thereof.

The present disclosure relates to the construction of chimeric antigen receptors targeting CD20, a preparation method of chimeric antigen receptor engineered T cells targeting CD20, and activity identification thereof.

In a first aspect of the disclosure, it provides a chimeric antigen receptor (CAR) (sequence), whose antigen binding domain (e.g., scFv) comprises an antibody heavy chain variable region as shown in SEQ ID NOs: 7 or 9 or 33 and an antibody light chain variable region as shown in SEQ ID NOs: 11 or 13 or 35.

In another embodiment, the antigen binding domain of the chimeric antigen receptor is as follows:

V_{H}-V_{L}
wherein V_{H} is an antibody heavy chain variable region; V_{L} is an antibody light chain variable region; and “-” is a linker peptide or a peptide bond.

In another embodiment, the amino acid sequence of the linker peptide is as shown in SEQ ID NO: 15.

In another embodiment, the amino acid sequence of V_{H} is as shown in SEQ ID NO: 7, and the amino acid sequence of V_{L} is as shown in SEQ ID NO: 11.

In another embodiment, the amino acid sequence of V_{H} is as shown in SEQ ID NO: 9, and the amino acid sequence of V_{L} is as shown in SEQ ID NO: 13.

In another embodiment, the amino acid sequence of V_{H} is as shown in SEQ ID NO: 33, and the amino acid sequence of V_{L} is shown in SEQ ID NO: 35.

In another embodiment, the structure of the chimeric antigen receptor is as follows:

L-V_{H}-V_{L}-H-TM-CS-CD3ζ
wherein,
L is an optional leader sequence (i.e., signal peptide);
H is a hinge region;
TM is a transmembrane domain;
CS is a co-stimulatory region or molecule derived from 4-1BB and/or CD28;
CD3ζ is a cytoplasmic signaling domain or sequence derived from CD3ζ;
V_{H}, V_{L}, and “-” are as described above, respectively.

In another embodiment, the sequence of L is as shown in SEQ ID NO: 27. In certain embodiments, the signal peptide may comprise an amino acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 27.

In another embodiment, the sequence of H is as shown in SEQ ID NO: 17 or 19. In certain embodiments, the hinge region may comprise an amino acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 17 or SEQ ID NO: 19.

In another embodiment, the sequence of TM comprises a transmembrane region derived from CD8a or CD28. For example, the sequence of TM is as shown in SEQ ID NO: 21 or 37. In certain embodiments, the transmembrane domain may comprise an amino acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 21 or SEQ ID NO: 37.

In another embodiment, the CS structure is: CD28-4-1BB, wherein CD28 is a co-stimulatory molecule derived from CD28; and 4-1BB is a co-stimulatory molecule derived from 4-1BB.

In another embodiment, the sequence of the co-stimulatory molecule derived from 4-1BB is as shown in SEQ ID NO: 23.

In another embodiment, the sequence of the co-stimulatory molecule derived from CD28 is as shown in SEQ ID NO: 39.

In certain embodiments, the co-stimulatory region may comprise an amino acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 23 or SEQ ID NO: 39.

In another embodiment, the sequence of CD3 is as shown in SEQ ID NO: 25. In certain embodiments, the cytoplasmic signaling domain may comprise an amino acid sequence about 80% to about 100%, about 85% to about 100%, about 90% to about 100%, about 95% to about 100%, at least or about 70%, at least or about 75%, at least or about 80%, at least or about 85%, at least or about 90%, at least or about 95%, at least or about 99%, at least or about 81%, at least or about 82%, at least or about 83%, at least or about 84%, at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, at least or about 99%, or about 100%, identical to the amino acid sequence set forth in SEQ ID NO: 25.

In another embodiment, the sequence of the chimeric antigen receptor is as shown in SEQ ID NOs: 1, 3, 5, 29, or 31.

In a second aspect of the invention, a nucleic acid molecule is provided, encoding the chimeric antigen receptor (CAR) of the first aspect of the disclosure.

In another embodiment, the nucleic acid molecule comprises a nucleic acid sequence encoding the hinge region selected from the group consisting of:
(a) a polynucleotide encoding a polypeptide as shown in SEQ ID NO: 17 or 19;
(b) a polynucleotide having a sequence as shown in SEQ ID NO: 18 or 20;
(c) a polynucleotide having a nucleotide sequence with ≥90% (preferably ≥95%) homologous to the sequence of SEQ ID NO: 18 or 20, and encoding the amino acid sequence of SEQ ID NO: 17 or 19;
(d) a polynucleotide complementary to the polynucleotide of any of (a) to (c).

In another embodiment, the nucleic acid molecule comprises a nucleic acid sequence encoding the CD8a transmembrane region selected from the group consisting of:
(a) a polynucleotide encoding a polypeptide as shown in SEQ ID NO: 21;
(b) a polynucleotide having a sequence as shown in SEQ ID NO: 22;
(c) a polynucleotide having a nucleotide sequence with ≥90% (preferably ≥95%) homologous to the sequence of SEQ ID NO: 22 and encoding the amino acid sequence of SEQ ID NO: 21;
(d) a polynucleotide complementary to the polynucleotide of any of (a) to (c).

In another embodiment, the nucleic acid molecule comprises a nucleic acid sequence encoding the 4-1BB (CD137) intracellular signal domain selected from the group consisting of:
(a) a polynucleotide encoding a polypeptide as shown in SEQ ID NO: 23;
(b) a polynucleotide having a sequence as shown in SEQ ID NO: 24;
(c) a polynucleotide having a nucleotide sequence with ≥90% (preferably ≥95%) homologous to the sequence of SEQ ID NO: 24 and encoding the amino acid sequence of SEQ ID NO: 23;
(d) a polynucleotide complementary to the polynucleotide of any of (a) to (c).

In another embodiment, the nucleic acid molecule comprises a nucleic acid sequence encoding the CD28 intracellular signal domain selected from the group consisting of:
(a) a polynucleotide encoding a polypeptide as shown in SEQ ID NO: 39;
(b) a polynucleotide having a sequence as shown in SEQ ID NO: 40;
(c) a polynucleotide having a nucleotide sequence with ≥90% (preferably ≥95%) homologous to the sequence of SEQ ID NO: 40 and encoding the amino acid sequence of SEQ ID NO: 39;
(d) a polynucleotide complementary to the polynucleotide of any of (a) to (c).

In another embodiment, the nucleic acid molecule comprises a nucleic acid sequence encoding the CD3ζ intracellular signal domain selected from the group consisting of:
(a) a polynucleotide encoding a polypeptide as shown in SEQ ID NO: 25;
(b) a polynucleotide having a sequence as shown in SEQ ID NO: 26;
(c) a polynucleotide having a nucleotide sequence with ≥90% (preferably ≥95%) homologous to the sequence of SEQ ID NO: 26 and encoding the amino acid sequence of SEQ ID NO: 25;
(d) a polynucleotide complementary to the polynucleotide of any of (a) to (c).

In another embodiment, the nucleic acid molecule comprises a nucleic acid sequence selected from the group consisting of:
(a) a polynucleotide encoding a polypeptide as shown in SEQ ID NOs: 1, 3, 5, 29 or 31;
(b) a polynucleotide having the sequence as shown in SEQ ID NOs: 2, 4, 6, 30 or 32;
(c) a polynucleotide having a nucleotide sequence with ≥95% (preferably ≥98%) homologous to the sequence of SEQ ID NOs: 2, 4, 6, 30 or 32, and encoding the amino acid sequence of SEQ ID NOs: 1, 3, 5, 29 or 31;
(d) a polynucleotide complementary to the polynucleotide of any of (a) to (c).

In another embodiment, the nucleic acid molecule is isolated.

In another embodiment, the nucleic acid molecule further comprises a polynucleotide encoding the leader sequence (directing sequence, signal peptide), and the amino acid sequence of the leader sequence is as shown in SEQ ID NO: 27; the polynucleotide encoding the leader sequence (signal peptide) may be as shown in SEQ ID NO: 28.

In another embodiment, the sequence of the nucleic acid molecule is as shown in SEQ ID NOs: 2, 4, 6, 30 or 32.

In a third aspect of the disclosure, it provides a vector, comprising the nucleic acid molecule of the second aspect of the invention.

In another embodiment, the vector is a lentiviral vector.

In a fourth aspect of the disclosure, it provides a host cell comprising the vector of the third aspect of the disclosure or having the exogenous nucleic acid molecule of the second aspect of the disclosure integrated into its genome.

In another embodiment, the cell is an isolated cell, and/or the cell is a genetically engineered cell.

In another embodiment, the cell is a mammalian cell.

In another embodiment, the cell is a T cell.

In a fifth aspect of the disclosure, it provides a pharmaceutical composition comprising a pharmaceutically acceptable carrier and the chimeric antigen receptor of the first aspect of the disclosure, the nucleic acid molecule of the second aspect of the disclosure, the vector of the third aspect of the disclosure, or the cell of the fourth aspect of the disclosure.

In a sixth aspect of the disclosure, it provides the use of the chimeric antigen receptor of the first aspect of the disclosure, the nucleic acid molecule of the second aspect of the disclosure, the vector of the third aspect of the disclosure, or the cell of the fourth aspect of the disclosure for the preparation of a medicine or a formulation for treating tumor or autoimmune disease.

In another embodiment, the autoimmune disease is an autoimmune disease caused by overexpression of B cells (such as lupus erythematosus).

In another embodiment, the tumor comprises CD20 positive tumor.

In a seventh aspect of the disclosure, it provides a method for treating a disease comprising administering an appropriate amount of the chimeric antigen receptor of the first aspect of the disclosure, the nucleic acid molecule of the second aspect of the disclosure, the vector of the third aspect of the disclosure, the cell of the fourth aspect of the disclosure, or the pharmaceutical composition of the fifth aspect of the disclosure, to a subject in need of treatment.

In another embodiment, the disease is tumor.

In an eighth aspect of the disclosure, it provides a method for preparing a CAR-T cell (CAR-modified T cell) expressing the chimeric antigen receptor of the first aspect of the disclosure. The method may comprise the steps of: transducing the nucleic acid molecule of the second aspect of the disclosure or the vector of the third aspect of the disclosure into a T cell, thereby obtaining the CAR-T cell.

It is to be understood that the various technical features of the present disclosure mentioned above and the various technical features specifically described hereinafter (as in the Examples) may be combined with each other within the scope of the present disclosure to constitute a new or preferred technical solution, which will not be repeated one by one herein.

### BRIEF DESCRIPTION OF THE DRAWINGS

FIG. 1 shows the structure of the chimeric antigen receptor targeting CD20. Each element of the designed CAR structure is shown in the figure, and the listed elements include: a leader sequence, an antigen recognition sequence (Ofatumumaband, Obinutuzumab, Rituximab), a hinge region, a transmembrane region, a co-stimulatory region, and a CD3ζ signaling region. CAR-T20.14, CAR-T20.13 and CAR-T20.16 are CAR structures constructed based on the antibody variable region sequences of Ofatumumab, Obinutuzumab and Rituxmab, respectively. CAR-T20.19 and CAR-20.20 are the mutant form of CAR-T20.14, having L235E-N297Q mutation in IgG4 Hinge-CH2-CH3 linker region. CAR-T20.20 is a third-generation chimeric antigen receptor structure with coding sequences of both CD28 and 4-1BB co-stimulatory signaling molecule.

FIGS. 2A-2B show detection of transfection efficiency of engineered T cell with chimeric antigen receptors targeting CD20. The expression level of the CAR gene-encoded protein on the surface of the T cell membrane in CAR-T20s cells cultured on day 7 (FIG. 2A) and day 11 (FIG. 2B) was identified by the Protein L method.

FIGS. 3A-3B. 1*10⁵ of NT, CART-20.13, CART-20.14 and CAR-T20.16 cells (cultured on day 6) were co-cultured respectively with CD20-positive RAJI and RAMOS tumor cell lines, and CD20-negative MOLT-4 tumor cell line in 200 μl GT-551 medium for 18 h in a ratio of 1:1. Then the expression level of CD137 on the surface of T cell membrane (FIG. 3A) and the secretion level of IFNγ in the co-culture supernatant (FIG. 3B) were detected.

FIG. 4 shows detection of apoptosis levels of tumor cells induced by CART-20. 1*10⁴ of CFSE-labeled CD20-negative (MOLT-4) or CD20-positive (RAJI, RAMOS) tumor cell lines were co-cultured respectively with NT, CART-20.13, CART-20.14 and CAR-T20.16 cells (cultured on day 11) in 200 μl GT-551 medium for 4 h according to the ratio as shown in figure. Then the cell pellet was collected by centrifugation. The cells were washed twice with PBS and stained for 30 min with Annexin V-APC dye in a ratio of 1:50 in 100 μl of dyeing solution. After washing with PBS for 1 time, the proportion of Annexin V positive cells in CFSE positive cells was analyzed on a flow cytometry. The results in figure show the statistical analysis of Annexin V positive cells in the corresponding co-culture samples.

FIGS. 5A-5C show identification of the activation ability in vitro of the third-generation chimeric antigen receptor and the chimeric antigen receptor with mutation in hinge region (which are constructed based on the sequence of Ofatumumaband antibody). The expression level of the CAR gene-encoded protein (FIG. 5A) on the surface of the T cell membrane in CAR-T20.14, CAR-T20.19 and CAR-T20.20 cells cultured on day 7 was identified by the Protein L method. 1*10⁵ of NT, CART-20.14, CART-20.19 and CAR-T20.20 cells (cultured on day 7) were cultured respectively with K562, K562 stable transfected cells of CD19 single positive, CD20 single positive, CD19 and CD20 double positive, and RAH target cell in 200 μl GT-551 medium for 18 h in a ratio of 1:1. Then the expression level of CD137 on the surface of T cell membrane (FIG. 5B) and the secretion level of IFNγ in the culture supernatant (FIG. 5C) were detected, respectively.

FIG. 6 shows the detection results of the ability of CAR-T20 cells to scavenge CD20-positive cells in vivo. The results indicate that CAR-T20.19 can effectively inhibit the in vivo expansion of CD20-positive tumor cells.

FIGS. 7A-7D show the screening of scFv for anti-CD20-CARs. FIG. 7A shows the structures of CAR-T20.1, CAR-T20.9 to CAR-T20.16. FIG. 7B shows the secretion levels of IFNγ. FIG. 7C shows the structures of CAR-T20.9, CAR-T20.12, CAR-T20.14, CAR-T20.17 to CAR-T20.19 (C-CAR066). FIG. 7B shows the secretion levels of IFNγ.

FIGS. 8A-8D show the lead selection for anti-CD20-CARs. FIG. 8A shows the structures of CAR-T20.17, CAR-T20.18 and CAR-T20.19 (C-CAR066). FIG. 8B shows the secretion levels of IFNγ. CAR-T20.19: CART20-OF(2nd). CAR-T20.17: CART20-LEU (3rd), which is the third-generation CAR with Leu-16 scFv. CAR-T20.18: CART20-LEU (2nd), which is the second-generation CAR with Leu-16 scFv. FIG. 8C shows the cytotoxicity of CAR-T20.17, CAR-T20.18 and CAR-T20.19 (C-CAR066). FIG. 8D shows in vivo anti-tumor efficacy of CAR-T20.17, CAR-T20.18 and CAR-T20.19 (C-CAR066).

FIG. 9A shows the structures of CAR-T20.19 (C-CAR066) and CAR-T20.29. FIG. 9B shows that CAR-T20.19 (C-CAR066) has the optimized V_{H}-V_{L}, scFv structure.

FIGS. 10A-10D show that CAR-T20.19 (C-CAR066) had superior in vivo anti-tumor activity. C-CAR011 is anti-CD19 41BB CAR with FMC63.

FIG. 11 shows an example of the CAR (C-CAR066) manufacture process. The process includes the usage of serum free media, and a functionally closed, highly automated system. Stars indicate improved processes.

FIG. 12 shows CAR066 Phase I clinical study design and flow chart. A Phase I, first in human, open-label study targeting/r B-NHL patients after failing CD19 CAR-T therapy conducted at two sites. Enrollment key eligibility criteria include 18-75 years old; measurable lesion, CD19 CAR-T failure, no active infection, adequate organ function and no CNS lesion. Objectives include the following. Primary objectives include incidence and severity of TEAEs (CTCAE V5.0 and ASTCT). Secondary objectives include ORR, DOR, PFS, OS (Lugano 2014). Exploratory objectives include CAR-T expansion and persistence.

FIG. 13 shows the CRS safety profile of C-CAR066.

FIG. 14A shows the C-CAR066 clinical response, including SD, PR, CR, and PD. SD: stable disease; PR: partial response; CR: complete response; PD: progressive disease. FIG. 14B shows the Kaplan Meyer estimation of progression-free survival (PFS). FIG. 14C shows the tumor burden (% change) in the seven patients.

FIG. 15A shows a case study where CR was achieved at 4 weeks with bulky disease. FIG. 15B shows the PET-CT images of the cancer lesions for one patient, patient No. 2.

FIGS. 16A-16F show C-CAR066 PK/PD profiles. FIG. 16A shows the changes of C-CAR066 CAR copies in the peripheral blood of the patients after CAR administration over time. FIG. 16B shows the changes of CD20+ B cell levels in the peripheral blood of the patients after CAR administration over time. FIG. 16C shows Cₘₐₓ levels in the blood of the patients after CAR administration. FIG. 16D shows AUC levels in the blood of the patients after CAR administration. FIG. 16E shows Tₘₐₓ levels in the blood of the patients after CAR administration. FIG. 16F shows Tₗₐₛₜ levels in the blood of the patients after CAR administration. Low dose: 2.0×10⁶ CAR-T cells/kg; mid dose: 3.0×10⁶ CAR-T cells/kg; high dose: 4.8×10⁶ CAR-T cells/kg.

FIG. 17 shows CD19/CD20 expression and PK/PD in C-CAR066 relapsed patients, including CAR-T expansion and B cell depletion in peripheral blood.

### DETAILED DESCRIPTION

The present disclosure provides for chimeric antigen receptors (CARs) targeting CD20. In certain embodiments, the CARs are based on three antibodies: Ofatumumab, Rituximab and Obinutuzumab. The present disclosure also provides for the in vitro activities and tumor cell killing efficacy of these chimeric antigen receptors. Studies have shown that the chimeric antigen receptors of the present disclosure target CD20-positive cells and can be used to treat a hematologic cancer including a B-cell malignancy such as acute lymphocytic leukemia (ALL), chronic lymphocytic leukemia (CLL), B-cell acute lymphoblastic leukemia (B-ALL), B-cell leukemia, or B cell lymphoma. The present CARs may be used to treat Hodgkin's lymphoma, non-Hodgkin's lymphoma, leukemia, and/or multiple myeloma (MM).

Chimeric antigen receptors targeting CD20 and the preparation and application thereof are provided. The extracellular antigen binding domain of the chimeric antigen receptor includes the antibody heavy chain variable region and the antibody light chain variable region. The experimental results show that the chimeric antigen receptor provided by the present disclosure shows significantly high killing ability against tumor cells.

In view of the differences in affinity, killing mechanism of therapeutic antibodies targeting CD20, as well as the significant effects of different transmembrane domains and intracellular domains on the activity of chimeric antigen receptor, a series of chimeric antigen receptors targeting CD20 were constructed in the present disclosure by combining various transmembrane and intracellular components with the amino acid sequences of the variable regions in various anti-CD20 antibodies. The expression of such chimeric antigen receptors in T cells (e.g., primary T cells) was completed. The detection method of receptor expression intensity was established. The ability of the CAR-T cells to recognize CD20 antigen in vitro and in vivo, as well as the difference in the activity of scavenging malignant tumors carrying CD20 antigen in vitro and in vivo were identified, providing a new effective method and preparation for the clinical application of CAR T in treating CD20 positive leukemia and lymphoma.

### Chimeric Antigen Receptors

The disclosure provides a chimeric antigen receptor (CAR) comprising an extracellular domain, a transmembrane domain, and an intracellular domain. The extracellular domain comprises a target-specific binding element (also known as an antigen binding region or domain). The intracellular domain includes a co-stimulatory (signaling) region and a ζ chain moiety. The co-stimulatory signaling region refers to a part of the intracellular domain that includes a co-stimulatory molecule. The co-stimulatory molecule is a cell surface molecule for efficient response of lymphocytes to antigens, rather than an antigen receptor or its ligand.

A linker can be incorporated between the extracellular domain and the transmembrane domain of the CAR, or between the cytoplasmic domain and the transmembrane domain of the CAR.

As used herein, the term “linker” generally refers to any oligopeptide or polypeptide that plays a role of linking the two components of the CAR. For example, a linker can link the transmembrane domain to the extracellular domain or the cytoplasmic domain in a polypeptide chain. The linker may comprise 0-300 amino acids, 2-100 amino acids, or 3-50 amino acids.

In certain embodiment, the extracellular domain of the CAR provided by the present disclosure comprises an antigen binding domain targeting CD20. When the CAR of the present disclosure is expressed in T cells, antigen recognition can be performed based on antigen binding specificity. When it binds to its cognate antigen, it affects a tumor cell so that the tumor cell fails to grow, is prompted to die, or otherwise is affected so that the tumor burden in a patient is diminished or eliminated. The antigen binding domain may be fused with an intracellular domain from one or more of a co-stimulatory molecule and a chain. In one embodiment, the antigen binding domain is fused with an intracellular domain of a combination of a 4-1BB signaling domain and/or a CD28 signaling domain, and a CD3 signaling domain.

In one embodiment, the CAR targeting CD20 comprises the specific signaling domain (e.g., the transmembrane region of CD8, the intracellular signal domains of CD137 and CD3 are in series). The signaling domain of the disclosure significantly increases anti-tumor activity and in vivo persistence of CAR-T cells compared to an otherwise identical CAR targeting CD20.

In one embodiment, the amino acid sequence of the chimeric antigen receptor (CAR) provided by the present disclosure is as follows.

| CAR-T20.13 | |
|---|---|
| (SEQ ID NO: 29) | |
| MALPVTALLL PLALLLHAAR PQVQLVQSGA EVKKPGSSVK VSCKASGYAF SYSWINWVRQ | 60 |
| APGQGLEWMG RIFPGDGDTD YNGKFKGRVT ITADKSTSTA YMELSSLRSE DTAVYYCARN | 120 |
| VFDGYWLVYW GQGTLVTVSS GGGGSGGGGS GGGGSDIVMT QTPLSLPVTP GEPASISCRS | 180 |
| SKSLLHSNGI TYLYWYLQKP GQSPQLLIYQ MSNLVSGVPD RFSGSGSGTD FTLKISRVEA | 240 |
| EDVGVYYCAQ NLEITYTEGG GTKVEIKRTV ESKYGPPCPP CPAPEFLGGP SVFLFPPKPK | 300 |
| DTLMISRTPE VTCVVVDVSQ EDPEVQFNWY VDGVEVHNAK TKPREEQFNS TYRVVSVLTV | 360 |
| LHQDWLNGKE YKCKVSNKGL PSSIEKTISK AKGQPREPQV YTLPPSQEEM TKNQVSLTCL | 420 |
| VKGFYPSDIA VEWESNGQPE NNYKTTPPVL DSDGSFFLYS RLTVDKSRWQ EGNVFSCSVM | 480 |
| HEALHNHYTQ KSLSLSLGKI YIWAPLAGTC GVLLLSLVIT LYCKRGRKKL LYIFKQPFMR | 540 |
| PVQTTQEEDG CSCRFPEEEE GGCELRVKFS RSADAPAYKQ GQNQLYNELN LGRREEYDVL | 600 |
| DKRRGRDPEM GGKPRRKNPQ EGLYNELQKD KMAEAYSEIG MKGERRRGKG HDGLYQGLST | 660 |
| ATKDTYDALH MQALPPR | 677 |
| The DNA sequence encoding CAR-T20.13 (SEQ ID NO: 30) may be as follows: | |
| atggccttac cagtgaccgc cttgctcctg ccgctggcct tgctgctcca cgccgccagg | 60 |
| ccgcaggtgc aattggtgca gtctggcgct gaagttaaga agcctgggag ttcagtgaag | 120 |
| gtctcctgca aggcttccgg atacgccttc agctattctt ggatcaattg ggtgcggcag | 180 |
| gcgcctggac aagggctcga gtggatggga cggatctttc ccggcgatgg ggatactgac | 240 |
| tacaatggga aattcaaggg cagagtcaca attaccgccg acaaatccac tagcacagcc | 300 |
| tatatggagc tgagcagcct gagatctgag gacacggccg tgtattactg tgcaagaaat | 360 |
| gtctttgatg gttactggct tgtttactgg ggccagggaa ccctggtcac cgtctcctca | 420 |
| ggtggcggtg gctcgggcgg tggtgggtcg ggtggcggcg gatctgatat cgtgatgacc | 480 |
| cagactccac tctccctgcc cgtcacccct ggagagcccg ccagcattag ctgcaggtct | 540 |
| agcaagagcc tcttgcacag caatggcatc acttatttgt attggtacct gcaaaagcca | 600 |
| gggcagtctc cacagctcct gatttatcaa atgtccaacc ttgtctctgg cgtccctgac | 660 |
| cggttctccg gctccgggtc aggcactgat ttcacactga aaatcagcag ggtggaggct | 720 |
| gaggatgttg gagtttatta ctgcgctcag aatctagaac ttccttacac cttcggcgga | 780 |
| gggaccaagg tggagatcaa acgtacggtg gagagcaagt acggaccgcc ctgcccccct | 840 |
| tgccctgccc ccgagttcct gggcggaccc agcgtgttcc tgttcccccc caagcccaag | 900 |
| gacaccctga tgatcagccg gacccccgag gtgacctgcg tggtggtgga cgtgagccag | 960 |
| gaagatcccg aggtccagtt caattggtac gtggacggcg tggaagtgca caacgccaag | 1020 |
| accaagccca gagaggaaca gttcaacagc acctaccggg tggtgtctgt gctgaccgtg | 1080 |
| ctgcaccagg actggctgaa cggcaaagaa tacaagtgca aggtgtccaa caagggcctg | 1140 |
| cccagcagca tcgaaaagac catcagcaag gccaagggcc agcctcgcga gccccaggtg | 1200 |
| tacaccctgc ctccctccca ggaagagatg accaagaacc aggtgtccct gacctgcctg | 1260 |
| gtgaagggct tctaccccag cgacatcgcc gtggagtggg agagcaacgg ccagcctgag | 1320 |
| aacaactaca agaccacccc tcccgtgctg gacagcgacg gcagcttctt cctgtacagc | 1380 |
| cggctgaccg tggacaagag ccggtggcag gaaggcaacg tctttagctg cagcgtgatg | 1440 |
| cacgaggccc tgcacaacca ctacacccag aagagcctga gcctgtccct gggcaagatc | 1500 |
| tacatctggg cgcccttggc cgggacttgt ggggtccttc tcctgtcact ggttatcacc | 1560 |
| ctttactgca aacggggcag aaagaaactc ctgtatatat tcaaacaacc atttatgaga | 1620 |
| ccagtacaaa ctactcaaga ggaagatggc tgtagctgcc gatttccaga agaagaagaa | 1680 |
| ggaggatgtg aactgagagt gaagttcagc aggagcgcag acgcccccgc gtacaagcag | 1740 |
| ggccagaacc agctctataa cgagctcaat ctaggacgaa gagaggagta cgatgttttg | 1800 |
| gacaagagac gtggccggga ccctgagatg gggggaaagc cgagaaggaa gaaccctcag | 1860 |
| gaaggcctgt acaatgaact gcagaaagat aagatggcgg aggcctacag tgagattggg | 1920 |
| atgaaaggcg agcgccggag gggcaagggg cacgatggcc tttaccaggg tctcagtaca | 1980 |
| gccaccaagg acacctacga cgcccttcac atgcaggccc tgccccctcg ctag | 2034 |
| The amino acid sequence of CAR-T20.14 (SEQ ID NO: 1): | |
| MALPVTALLL PLALLLHAAR PEVQLVESGG GLVQPGRSLR LSCAASGFTF NDYAMHWVRQ | 60 |
| APGKGLEWVS TISWNSGSIG YADSVKGRFT ISRDNAKKSL YLQMNSLRAE DTALYYCAKD | 120 |
| IQYGNYYYGM DVWGQGTTVT VSSGGGGSGG GGSGGGGSEI VLTQSPATLS LSPGERATLS | 180 |
| CRASQSVSSY LAWYQQKPGQ APRLLIYDAS NRATGIPARF SGSGSGTDFT LTISSLEPED | 240 |
| FAVYYCQQRS NWPITFGQGT RLEIKESKYG PPCPPCPAPE FLGGPSVFLF PPKPKDTLMI | 300 |
| SRTPEVTCVV VDVSQEDPEV QFNWYVDGVE VHNAKTKPRE EQFNSTYRVV SVLTVLHQDW | 360 |
| LNGKEYKCKV SNKGLPSSIE KTISKAKGQP REPQVYTLPP SQEEMTKNQV SLTCLVKGFY | 420 |
| PSDIAVEWES NGQPENNYKT TPPVLDSDGS FFLYSRLTVD KSRWQEGNVF SCSVMHEALH | 480 |
| NHYTQKSLSL SLGKIYIWAP LAGTCGVLLL SLVITLYCKR GRKKLLYIFK QPFMRPVQTT | 540 |
| QEEDGCSCRF PEEEEGGCEL RVKFSRSADA PAYKQGQNQL YNELNLGRRE EYDVLDKRRG | 600 |
| RDPEMGGKPR RKNPQEGLYN ELQKDKMAEA YSEIGMKGER RRGKGHDGLY QGLSTATKDT | 660 |
| YDALHMQALP PR | 672 |
| The DNA sequence encoding CAR-T20.14 (SEQ ID NO: 2) is as follows: | |
| atggccttac cagtgaccgc cttgctcctg ccgctggcct tgctgctcca cgccgccagg | 60 |
| ccggaagtgc agctggtgga gtctggggga ggcttggtac agcctggcag gtccctgaga | 120 |
| ctctcctgtg cagcctctgg attcaccttt aatgattatg ccatgcactg ggtccggcaa | 180 |
| gctccaggga agggcctgga gtgggtctca actattagtt ggaatagtgg ttccataggc | 240 |
| tatgcggact ctgtgaaggg ccgattcacc atctccagag acaacgccaa gaagtccctg | 300 |
| tatctgcaaa tgaacagtct gagagctgag gacacggcct tgtattactg tgcaaaagat | 360 |
| atacagtacg gcaactacta ctacggtatg gacgtctggg gccaagggac cacggtcacc | 420 |
| gtctcctcag gtggcggtgg ctcgggcggt ggtgggtcgg gtggcggcgg atctgaaatt | 480 |
| gtgttgacac agtctccagc caccctgtct ttgtctccag gggaaagagc caccctctcc | 540 |
| tgcagggcca gtcagagtgt tagcagctac ttagcctggt accaacagaa acctggccag | 600 |
| gctcccaggc tcctcatcta tgatgcatcc aacagggcca ctggcatccc agccaggttc | 660 |
| agtggcagtg ggtctgggac agacttcact ctcaccatca gcagcctaga gcctgaagat | 720 |
| tttgcagttt attactgtca gcagcgtagc aactggccga tcaccttcgg ccaagggaca | 780 |
| cgactggaga ttaaagagag caagtacgga ccgccctgcc ccccttgccc tgcccccgag | 840 |
| ttcctgggcg gacccagcgt gttcctgttc ccccccaagc ccaaggacac cctgatgatc | 900 |
| agccggaccc ccgaggtgac ctgcgtggtg gtggacgtga gccaggaaga tcccgaggtc | 960 |
| cagttcaatt ggtacgtgga cggcgtggaa gtgcacaacg ccaagaccaa gcccagagag | 1020 |
| gaacagttca acagcaccta ccgggtggtg tctgtgctga ccgtgctgca ccaggactgg | 1080 |
| ctgaacggca aagaatacaa gtgcaaggtg tccaacaagg gcctgcccag cagcatcgaa | 1140 |
| aagaccatca gcaaggccaa gggccagcct cgcgagcccc aggtgtacac cctgcctccc | 1200 |
| tcccaggaag agatgaccaa gaaccaggtg tccctgacct gcctggtgaa gggcttctac | 1260 |
| cccagcgaca tcgccgtgga gtgggagagc aacggccagc ctgagaacaa ctacaagacc | 1320 |
| acccctcccg tgctggacag cgacggcagc ttcttcctgt acagccggct gaccgtggac | 1380 |
| aagagccggt ggcaggaagg caacgtcttt agctgcagcg tgatgcacga ggccctgcac | 1440 |
| aaccactaca cccagaagag cctgagcctg tccctgggca agatctacat ctgggcgccc | 1500 |
| ttggccggga cttgtggggt ccttctcctg tcactggtta tcacccttta ctgcaaacgg | 1560 |
| ggcagaaaga aactcctgta tatattcaaa caaccattta tgagaccagt acaaactact | 1620 |
| caagaggaag atggctgtag ctgccgattt ccagaagaag aagaaggagg atgtgaactg | 1680 |
| agagtgaagt tcagcaggag cgcagacgcc cccgcgtaca agcagggcca gaaccagctc | 1740 |
| tataacgagc tcaatctagg acgaagagag gagtacgatg ttttggacaa gagacgtggc | 1800 |
| cgggaccctg agatgggggg aaagccgaga aggaagaacc ctcaggaagg cctgtacaat | 1860 |
| gaactgcaga aagataagat ggcggaggcc tacagtgaga ttgggatgaa aggcgagcgc | 1920 |
| cggaggggca aggggcacga tggcctttac cagggtctca gtacagccac caaggacacc | 1980 |
| tacgacgccc ttcacatgca ggccctgccc cctcgctag | 2019 |
| The amino acid sequence of CAR-T20.16 | |
| (SEQ ID NO: 3) | |
| MALPVTALLL PLALLLHAAR PQVQLQQPGA ELVKPGASVK MSCKASGYTF TSYNMHWVKQ | 60 |
| TPGRGLEWIG AIYPGNGDTS YNQKFKGKAT LTADKSSSTA YMQLSSLTSE DSAVYYCARS | 120 |
| TYYGGDWYFN VWGAGTTVTV SAGGGGSGGG GSGGGGSQIV LSQSPAILSA SPGEKVTMTC | 180 |
| RASSSVSYTH WFQQKPGSSP KPWIYATSNL ASGVPVRFSG SGSGTSYSLT ISRVEAEDAA | 240 |
| TYYCQQWTSN PPTFGGGTKL EIKESKYGPP CPPCPAPEFL GGPSVFLFPP KPKDTLMISR | 300 |
| TPEVTCVVVD VSQEDPEVQF NWYVDGVEVH NAKTKPREEQ FNSTYRVVSV LTVLHQDWLN | 360 |
| GKEYKCKVSN KGLPSSIEKT ISKAKGQPRE PQVYTLPPSQ EEMTKNQVSL TCLVKGFYPS | 420 |
| DIAVEWESNG QPENNYKTTP PVLDSDGSFF LYSRLTVDKS RWQEGNVESC SVMHEALHNH | 480 |
| YTQKSLSLSL GKIYIWAPLA GTCGVLLLSL VITLYCKRGR KKLLYIFKQP FMRPVQTTQE | 540 |
| EDGCSCREPE LEIGGCELRV KESRSADAPA YKQGQNQLYN ELNLGRRLEY DVLDKRRGRD | 600 |
| PEMGGKPRRK NPQEGLYNEL QKDKMAEAYS EIGMKGERRR GKGHDGLYQG LSTATKDTYD | 660 |
| ALHMQALPPR | 670 |
| The DNA sequence encoding CAR-T20.16 (SEQ ID NO: 4) is as follows: | |
| ATGGCCTTAC CAGTGACCGC CTTGCTCCTG CCGCTGGCCT TGCTGCTCCA CGCCGCCAGG | 60 |
| CCGCAGGTGC AGTTGCAACA GCCTGGAGCT GAGTTGGTGA AGCCTGGTGC TTCTGTGAAG | 120 |
| ATGTCTTGTA AGGCTTCTGG ATACACATTC ACTTCTTACA ACATGCACTG GGTGAAGCAG | 180 |
| ACTCCTGGTA GGGGTTTGGA GTGGATCCGA GCTATCTACC CAGGAAACGG AGACACATCT | 240 |
| TACAACCAGA AGTTCAAGGG TAAGGCTACA TTGACTGCTG ACAAGTCTTC ATCTACTGCT | 300 |
| TACATGCAAT TGTCTTCTTT GACATCTGAG GACTCTGCAG TTTACTACTG CGCTAGGTCT | 360 |
| ACATACTACG GAGGTGACTG GTACTTCAAC GTGTGGGGAG CAGGTACCAC GGTCACTGTC | 420 |
| TCTGCAGGTG GAGGTGGATC TGGAGGAGGA GGATCTGGTG GAGGAGGTTC TCAAATTGTT | 480 |
| CTCTCCCAGT CTCCAGCAAT CCTGTCAGCT TCTCCTGGAG AGAAGGTGAC TATGACTTGC | 540 |
| AGGGCTTCTT CATCTGTTTC TTACATCCAC TGGTTCCAGC AGAAGCCTGG TTCTTCACCT | 600 |
| AAGCCTTGGA TCTACGCTAC ATCTAACTTG GCATCTGGAG TGCCTGTGAG GTTCTCTGGT | 660 |
| TCTGGTTCAG GTACTTCTTA CTCTTTGACA ATCTCTAGGG TGGAGGCTGA GGACGCTGCT | 720 |
| ACTTACTACT GCCAGCAGTG GACATCTAAC CCTCCAACAT TCGGAGGTGG TACTAAGTTG | 780 |
| GAGATCAAGG AGAGCAAGTA CGGACCGCCC TGCCCCCCTT GCCCTGCCCC CGAGTTCCTG | 840 |
| GGCGGACCCA GCGTGTTCCT GTTCCCCCCC AAGCCCAAGG ACACCCTGAT GATCAGCCGG | 900 |
| ACCCCCGAGG TGACCTGCGT GGTGGTGGAC GTGAGCCAGG AAGATCCCGA GGTCCAGTTC | 960 |
| AATTGGTACG TGGACGGCGT GGAAGTGCAC AACGCCAAGA CCAAGCCCAG AGAGGAACAG | 1020 |
| TTCAACAGCA CCTACCGGGT GGTGTCTGTG CTGACCGTGC TGCACCAGGA CTGGCTGAAC | 1080 |
| GGCAAAGAAT ACAAGTGCAA GGTGTCCAAC AAGGGCCTGC CCAGCAGCAT CGAAAAGACC | 1140 |
| ATCAGCAAGG CCAAGGGCCA GCCTCGCGAG CCCCAGGTGT ACACCCTGCC TCCCTCCCAG | 1200 |
| GAAGAGATGA CCAAGAACCA GGTGTCCCTG ACCTGCCTGG TGAAGGGCTT CTACCCCAGC | 1260 |
| GACATCGCCG TGGAGTGGGA GAGCAACGGC CAGCCTGAGA ACAACTACAA GACCACCCCT | 1320 |
| CCCGTGCTGG ACAGCGACGG CAGCTTCTTC CTGTACAGCC GGCTGACCGT GGACAAGAGC | 1380 |
| CGGTGGCAGG AAGGCAACGT CTTTAGCTGC AGCGTGATGC ACGAGGCCCT GCACAACCAC | 1440 |
| TACACCCAGA AGAGCCTGAG CCTGTCCCTG GGCAAGATCT ACATCTGGGC GCCCTTGGCC | 1500 |
| GGGACTTGTG GGGTCCTTCT CCTGTCACTG GTTATCACCC TTTACTGCAA ACGGGGCAGA | 1560 |
| AAGAAACTCC TGTATATATT CAAACAACCA TTTATGAGAC CAGTACAAAC TACTCAAGAG | 1620 |
| GAAGATGGCT GTAGCTGCCG ATTTCCAGAA GAAGAAGAAG GAGGATGTGA ACTGAGAGTG | 1680 |
| AAGTTCAGCA GGAGCGCAGA CGCCCCCGCG TACAAGCAGG GCCAGAACCA GCTCTATAAC | 1740 |
| GAGCTCAATC TAGGACGAAG AGAGGAGTAC GATGTTTTGG ACAAGAGACG TGGCCGGGAC | 1800 |
| CCTGAGATGG GGGGAAAGCC GAGAAGGAAG AACCCTCAGG AAGGCCTGTA CAATGAACTG | 1860 |
| CAGAAAGATA AGATGGCGGA GGCCTACAGT GAGATTGGGA TGAAAGGCGA GCGCCGGAGG | 1920 |
| GGCAAGGGGC ACGATGGCCT TTACCAGGGT CTCAGTACAG CCACCAAGGA CACCTACGAC | 1980 |
| GCCCTTCACA TGCAGGCCCT GCCCCCTCGC TAG | 2013 |

In another embodiment, the amino acid sequence of the chimeric antigen receptor (CAR) provided by the disclosure is as follows.

| The amino acid sequence of CAR-T20.19 | |
|---|---|
| (SEQ ID NO: 5) | |
| MALPVTALLL PLALLLHAAR PEVQLVESGG GLVQPGRSLR LSCAASGFTF NDYAMHWVRQ | 60 |
| APGKGLEWVS TISWNSGSIG YADSVKGRFT ISRDNAKKSL YLQMNSLRAE DTALYYCAKD | 120 |
| IQYGNYYYGM DVWGQGTTVT VSSGGGGSGG GGSGGGGSEI VLTQSPATLS LSPGERATLS | 180 |
| CRASQSVSSY LAWYQQKPGQ APRLITYDAS NRATGIPARF SGSGSGTDFT LTISSLEPED | 240 |
| FAVYYCQQRS NWPITFGQGT RLEIKESKYG PPCPPCPAPE FEGGPSVFLF PPKPKDTLMI | 300 |
| SRTPEVTCVV VDVSQEDPEV QFNWYVDGVE VHNAKTKPRE EQFQSTYRVV SVLTVLHQDW | 360 |
| LNGKEYKCKV SNKGLPSSIE KTISKAKGQP REPQVYTLPP SQEEMTKNQV SLTCLVKGFY | 420 |
| PSDIAVEWES NGQPENNYKT TPPVLDSDGS FFLYSRLTVD KSRWQEGNVF SCSVMHEALH | 480 |
| NHYTQKSLSL SLGKIYIWAP LAGTCGVLLL SLVITLYCKR GRKKLLYIFK QPFMRPVQTT | 540 |
| QEEDGCSCRF PEEEEGGCEL RVKFSRSADA PAYKQGQNQL YNELNLGRRE EYDVLDKRRG | 600 |
| RDPEMGGKPR RKNPQEGLYN ELQKDKMAEA YSEIGMKGER RRGKGHDGLY QGLSTATKDT | 660 |
| YDALHMQALP PR | 672 |
| The DNA sequence encoding CAR-T20.19 (SEQ ID NO: 6) is as follows: | |
| atggccttac cagtgaccgc cttgctcctg ccgctggcct tgctgctcca cgccgccagg | 60 |
| ccggaagtgc agctggtgga gtctggggga ggcttggtac agcctggcag gtccctgaga | 120 |
| ctctcctgtg cagcctctgg attcaccttt aatgattatg ccatgcactg ggtccggcaa | 180 |
| gctccaggga agggcctgga gtgggtctca actattagtt ggaatagtgg ttccataggc | 240 |
| tatgcggact ctgtgaaggg ccgattcacc atctccagag acaacgccaa gaagtccctg | 300 |
| tatctgcaaa tgaacagtct gagagctgag gacacggcct tgtattactg tgcaaaagat | 360 |
| atacagtacg gcaactacta ctacggtatg gacgtctggg gccaagggac cacggtcacc | 420 |
| gtctcctcag gtggcggtgg ctcgggcggt ggtgggtcgg gtggcggcgg atctgaaatt | 480 |
| gtgttgacac agtctccagc caccctgtct ttgtctccag gggaaagagc caccctctcc | 540 |
| tgcagggcca gtcagagtgt tagcagctac ttagcctggt accaacagaa acctggccag | 600 |
| gctcccaggc tcctcatcta tgatgcatcc aacagggcca ctggcatccc agccaggttc | 660 |
| agtggcagtg ggtctgggac agacttcact ctcaccatca gcagcctaga gcctgaagat | 720 |
| tttgcagttt attactgtca gcagcgtagc aactggccga tcaccttcgg ccaagggaca | 780 |
| cgactggaga ttaaagagag caagtacgga ccgccctgcc ccccttgccc tgcccccgag | 840 |
| ttcgagggcg gacccagcgt gttcctgttc ccccccaagc ccaaggacac cctgatgatc | 900 |
| agccggaccc ccgaggtgac ctgcgtggtg gtggacgtga gccaggaaga tcccgaggtc | 960 |
| cagttcaatt ggtacgtgga cggcgtggaa gtgcacaacg ccaagaccaa gcccagagag | 1020 |
| gaacagttcc aaagcaccta ccgggtggtg tctgtgctga ccgtgctgca ccaggactgg | 1080 |
| ctgaacggca aagaatacaa gtgcaaggtg tccaacaagg gcctgcccag cagcatcgaa | 1140 |
| aagaccatca gcaaggccaa gggccagcct cgcgagcccc aggtgtacac cctgcctccc | 1200 |
| tcccaggaag agatgaccaa gaaccaggtg tccctgacct gcctggtgaa gggcttctac | 1260 |
| cccagcgaca tcgccgtgga gtgggagagc aacggccagc ctgagaacaa ctacaagacc | 1320 |
| acccctcccg tgctggacag cgacggcagc ttcttcctgt acagccggct gaccgtggac | 1380 |
| aagagccggt ggcaggaagg caacgtcttt agctgcagcg tgatgcacga ggccctgcac | 1440 |
| aaccactaca cccagaagag cctgagcctg tccctgggca agatctacat ctgggcgccc | 1500 |
| ttggccggga cttgtggggt ccttctcctg tcactggtta tcacccttta ctgcaaacgg | 1560 |
| ggcagaaaga aactcctgta tatattcaaa caaccattta tgagaccagt acaaactact | 1620 |
| caagaggaag atggctgtag ctgccgattt ccagaagaag aagaaggagg atgtgaactg | 1680 |
| agagtgaagt tcagcaggag cgcagacgcc cccgcgtaca agcagggcca gaaccagctc | 1740 |
| tataacgagc tcaatctagg acgaagagag gagtacgatg ttttggacaa gagacgtggc | 1800 |
| cgggaccctg agatgggggg aaagccgaga aggaagaacc ctcaggaagg cctgtacaat | 1860 |
| gaactgcaga aagataagat ggcggaggcc tacagtgaga ttgggatgaa aggcgagcgc | 1920 |
| cggaggggca aggggcacga tggcctttac cagggtctca gtacagccac caaggacacc | 1980 |
| tacgacgccc ttcacatgca ggccctgccc cctcgctag | 2019 |

In one embodiment, the amino acid sequence of the chimeric antigen receptor (CAR) provided by the invention is as follows.

| The amino acid sequence of CAR-T20.20 | |
|---|---|
| (SEQ ID NO: 31) | |
| MALPVTALLL PLALLLHAAR PEVQLVESGG GLVQPGRSLR LSCAASGFTF NDYAMHWVRQ | 60 |
| APGKGLEWVS TISWNSGSIG YADSVKGRFT ISRDNAKKSL YLQMNSLRAE DTALYYCAKD | 120 |
| IQYGNYYYGM DVWGQGTTVT VSSGGGGSGG GGSGGGGSEI VLTQSPATLS LSPGERATLS | 180 |
| CRASQSVSSY LAWYQQKPGQ APRLLIYDAS NRATGIPARF SGSGSGTDFT LTISSLEPED | 240 |
| FAVYYCQQRS NWPITFGQGT RLEIKESKYG PPCPPCPAPE FEGGPSVFLF PPKPKDTLMI | 300 |
| SRTPEVTCVV VDVSQEDPEV QFNWYVDGVE VHNAKTKPRE EQFQSTYRVV SVLTVLHQDW | 360 |
| LNGKEYKCKV SNKGLPSSIE KTISKAKGQP REPQVYTLPP SQEEMTKNQV SLTCLVKGFY | 420 |
| PSDIAVEWES NGQPENNYKT TPPVLDSDGS FFLYSRLTVD KSRWQEGNVF SCSVMHEALH | 480 |
| NHYTQKSLSL SLGKFWVLVV VGGVLACYSL LVTVAFIIFW VRSKRSRLLH SDYMNMTPRR | 540 |
| PGPTRKHYQP YAPPRDFAAY RSKRGRKKLL YIFKQPFMRP VQTTQEEDGC SCRFPEEEEG | 600 |
| GCELRVKFSR SADAPAYKQG QNQLYNELNL GRREEYDVLD KRRGRDPEMG GKPRRKNPQE | 660 |
| GLYNELQKDK MAEAYSEIGM KGERRRGKGH DGLYQGLSTA TKDTYDALHM QALPPR | 716 |

| The coding DNA sequence of CAR-T20.20 (SEQ ID No: 32) is as follows: | |
|---|---|
| atggccttac cagtgaccgc cttgctcctg ccgctggcct tgctgctcca cgccgccagg | 60 |
| ccggaagtgc agctggtgga gtctggggga ggcttggtac agcctggcag gtccctgaga | 120 |
| ctctcctgtg cagcctctgg attcaccttt aatgattatg ccatgcactg ggtccggcaa | 180 |
| gctccaggga agggcctgga gtgggtctca actattagtt ggaatagtgg ttccataggc | 240 |
| tatgcggact ctgtgaaggg ccgattcacc atctccagag acaacgccaa gaagtccctg | 300 |
| tatctgcaaa tgaacagtct gagagctgag gacacggcct tgtattactg tgcaaaagat | 360 |
| atacagtacg gcaactacta ctacggtatg gacgtctggg gccaagggac cacggtcacc | 420 |
| gtctcctcag gtggcggtgg ctcgggcggt ggtgggtcgg gtggcggcgg atctgaaatt | 480 |
| gtgttgacac agtctccagc caccctgtct ttgtctccag gggaaagagc caccctctcc | 540 |
| tgcagggcca gtcagagtgt tagcagctac ttagcctggt accaacagaa acctggccag | 600 |
| gctcccaggc tcctcatcta tgatgcatcc aacagggcca ctggcatccc agccaggttc | 660 |
| agtggcagtg ggtctgggac agacttcact ctcaccatca gcagcctaga gcctgaagat | 720 |
| tttgcagttt attactgtca gcagcgtagc aactggccga tcaccttcgg ccaagggaca | 780 |
| cgactggaga ttaaagagag caagtacgga ccgccctgcc ccccttgccc tgcccccgag | 840 |
| ttcgagggcg gacccagcgt gttcctgttc ccccccaagc ccaaggacac cctgatgatc | 900 |
| agccggaccc ccgaggtgac ctgcgtggtg gtggacgtga gccaggaaga tcccgaggtc | 960 |
| cagttcaatt ggtacgtgga cggcgtggaa gtgcacaacg ccaagaccaa gcccagagag | 1020 |
| gaacagttcc aaagcaccta ccgggtggtg tctgtgctga ccgtgctgca ccaggactgg | 1080 |
| ctgaacggca aagaatacaa gtgcaaggtg tccaacaagg gcctgcccag cagcatcgaa | 1140 |
| aagaccatca gcaaggccaa gggccagcct cgcgagcccc aggtgtacac cctgcctccc | 1200 |
| tcccaggaag agatgaccaa gaaccaggtg tccctgacct gcctggtgaa gggcttctac | 1260 |
| cccagcgaca tcgccgtgga gtgggagagc aacggccagc ctgagaacaa ctacaagacc | 1320 |
| acccctcccg tgctggacag cgacggcagc ttcttcctgt acagccggct gaccgtggac | 1380 |
| aagagccggt ggcaggaagg caacgtcttt agctgcagcg tgatgcacga ggccctgcac | 1440 |
| aaccactaca cccagaagag cctgagcctg tccctgggca agttttgggt gctggtggtg | 1500 |
| gttggtggag tcctggcttg ctatagcttg ctagtaacag tggcctttat tattttctgg | 1560 |
| gtgaggagta agaggagcag gctcctgcac agtgactaca tgaacatgac tccccgccgc | 1620 |
| cccgggccca cccgcaagca ttaccagccc tatgccccac cacgcgactt cgcagcctat | 1680 |
| cgctccaaac ggggcagaaa gaaactcctg tatatattca aacaaccatt tatgagacca | 1740 |
| gtacaaacta ctcaagagga agatggctgt agctgccgat ttccagaaga agaagaagga | 1800 |
| ggatgtgaac tgagagtgaa gttcagcagg agcgcagacg cccccgcgta caagcagggc | 1860 |
| cagaaccagc tctataacga gctcaatcta ggacgaagag aggagtacga tgttttggac | 1920 |
| aagagacgtg gccgggaccc tgagatgggg ggaaagccga gaaggaagaa ccctcaggaa | 1980 |
| ggcctgtaca atgaactgca gaaagataag atggcggagg cctacagtga gattgggatg | 2040 |
| aaaggcgagc gccggagggg caaggggcac gatggccttt accagggtct cagtacagcc | 2100 |
| accaaggaca cctacgacgc ccttcacatg caggccctgc cccctcgcta a | 2151 |

### Antigen Binding Region (Domain)

In one embodiment, the CAR of the disclosure comprises a target-specific binding element referred to as antigen binding region or domain. The antigen binding domain of the present CAR is a specific binding element targeting CD20.

In one embodiment, the antigen binding domain comprises a heavy chain variable region and a light chain variable region of an anti-CD20 antibody.

In another embodiment, the amino acid sequence of the heavy chain variable region of the Ofatumumab antibody is as follows:

(SEQ ID NO: 7)
EVQLVESGGG LVQPGRSLRL SCAASGFTFN DYAMHWVRQA PGKGLEWVST ISWNSGSIGY 60
ADSVKGRFTI SRDNAKKSLY LQMNSLRAED TALYYCAKDI QYGNYYYGMD VWGQGTTVTV 120
SS 122

The DNA sequence encoding the heavy chain variable region of the Ofatumumab antibody is as follows:

(SEQ ID NO: 8)
GAAGTGCAGC TGGTGGAGTC TGGGGGAGGC TTGGTACAGC CTGGCAGGTC CCTGAGACTC 60
TCCTGTGCAG CCTCTGGATT CACCTTTAAT GATTATGCCA TGCACTGGGT CCGGCAAGCT 120
CCAGGGAAGG GCCTGGAGTG GGTCTCAACT ATTAGTTGGA ATAGTGGTTC CATAGGCTAT 180
GCGGACTCTG TGAAGGGCCG ATTCACCATC TCCAGAGACA ACGCCAAGAA GTCCCTGTAT 240
CTGCAAATGA ACAGTCTGAG AGCTGAGGAC ACGGCCTTGT ATTACTGTGC AAAAGATATA 300
CAGTACGGCA ACTACTACTA CGGTATGGAC GTCTGGGGCC AAGGGACCAC GGTCACCGTC 360
TCCTCA 366

The amino acid sequence of the heavy chain variable region of the Rituximab antibody is as follows:

(SEQ ID NO: 9)
QVQLQQPGAE LVKPGASVKM SCKASGYTFT SYNMHWVKQT PGRGLEWIGA IYPGNGDTSY 60
NQKFKGKATL TADKSSSTAY MQLSSLTSED SAVYYCARST YYGGDWYFNV WGAGTTVTVS 120
A 121

The DNA sequence encoding the heavy chain variable region of the Rituximab antibody is as follows:

(SEQ ID NO: 10)
CAGGTGCAGT TGCAACAGCC TGGAGCTGAG TTGGTGAAGC CTGGTGCTTC TGTGAAGATG 60
TCTTGTAAGG CTTCTGGATA CACATTCACT TCTTACAACA TGCACTGGGT GAAGCAGACT 120
CCTGGTAGGG GTTTGGAGTG GATCGGAGCT ATCTACCCAG GAAACGGAGA CACATCTTAC 180
AACCAGAAGT TCAAGGGTAA GGCTACATTG ACTGCTGACA AGTCTTCATC TACTGCTTAC 240
ATGCAATTGT CTTCTTTGAC ATCTGAGGAC TCTGCAGTTT ACTACTGCGC TAGGTCTACA 300
TACTACGGAG GTGACTGGTA CTTCAACGTG TGGGGAGCAG GTACCACGGT CACTGTCTCT 360
GCA. 363

Further, the amino acid sequence of the heavy chain variable region of the Obinutuzumab antibody used in the present disclosure is as follows:

(SEQ ID NO: 33)
QVQLVQSGAE VKKPGSSVKV SCKASGYAFS YSWINWVRQA PGQGLEWMGR IFPGDGDTDY 60
NGKFKGRVTI TADKSTSTAY MELSSLRSED TAVYYCARNV FDGYWLVYWG QGTLVTVSS 119

The DNA sequence encoding the heavy chain variable region of the Obinutuzumab antibody is as follows:

(SEQ ID NO: 34)
caggtgcaat tggtgcagtc tggcgctgaa gttaagaagc ctgggagttc agtgaaggtc 60
tcctgcaagg cttccggata cgccttcagc tattcttgga tcaattgggt gcggcaggcg 120
cctggacaag ggctcgagtg gatgggacgg atctttcccg gcgatgggga tactgactac 180
aatgggaaat tcaagggcag agtcacaatt accgccgaca aatccactag cacagcctat 240
atggagctga gcagcctgag atctgaggac acggccgtgt attactgtgc aagaaatgtc 300
tttgatggtt actggcttgt ttactggggc cagggaaccc tggtcaccgt ctcctca 357

In another embodiment, the amino acid sequence of the light chain variable region of the Ofatumumaband antibody is as follows:

(SEQ ID NO: 11)
EIVLTQSPAT LSLSPGERAT LSCRASQSVS SYLAWYQQKP GQAPRLLTYD ASNRATGIPA 60
RFSCSGSGTD FTLTISSLEP EDFAVYYCQQ RSNWPITFGQ GTRLEIK 107

The DNA sequence of Ofatumumaband antibody is as follows:

(SEQ ID NO: 12)
GAAATTGTGT TGACACAGTC TCCAGCCACC CTGTCTTTGT CTCCAGGGGA AAGAGCCACC 60
CTCTCCTGCA GGGCCAGTCA GAGTGTTAGC AGCTACTTAG CCTGGTACCA ACAGAAACCT 120
GGCCAGGCTC CCAGGCTCCT CATCTATGAT GCATCCAACA GGGCCACTGG CATCCCAGCC 180
AGGTTCAGTG GCAGTGGGTC TGGGACAGAC TTCACTCTCA CCATCAGCAG CCTAGAGCCT 240
GAAGATTTTG CAGTTTATTA CTGTCAGCAG CGTAGCAACT GGCCGATCAC CTTCGGCCAA 300
GGGACACGAC TGGAGATTAA A 321

The anti-CD20 CAR comprises an anti-CD20 antigen-binding region which comprises a light chain variable region (V_{L}) and a heavy chain variable region (V_{H}). V_{L} comprises three complementarity determining regions (CDRs), LCDR1, LCDR2 and LCDR3, and V_{H} comprises three CDRs, HCDR1, HCDR2 and HCDR3.

The CDRs of Ofatumumab are as follows. V_{H} comprises three CDRs: CDR-H1 (HCDR1), CDR-H2 (HCDR2) and CDR-H3 (HCDR3); V_{L} comprises three CDRs: CDR-L1 (LCDR1), CDR-L2 (LCDR2) and CDR-L3 (LCDR3).
CDR-H1: NDYAMH (SEQ ID NO: 41)
CDR-H2: TISWNSGSIGYADSVKG (SEQ ID NO: 42)
CDR-H3: DIQYGNYYYGMDV (SEQ ID NO: 43)
CDR-L1: RASQSVSSYLA (SEQ ID NO: 44)
CDR-L2: DASNRAT (SEQ ID NO: 45)
CDR-L3: QQRSNWPIT (SEQ ID NO: 46)

The amino acid sequence of the light chain variable region of the Rituximab antibody is as follows:

(SEQ ID NO: 13)
QIVLSQSPAI LSASPGEKVT MTCRASSSVS YTHWFQQKPG SSPKPWIYAT SNLASGVPVR 60
FSGSGSGTSY SLTISRVEAE DAATYYCQQW TSNPPTFGGG TKLEIK 106

The DNA sequences encoding the light chain (VL) of single-chain variable region derived from the Rituximab antibody is:

(SEQ ID NO: 14)
CAAATTGTTC TCTCCCAGTC TCCAGCAATC CTGTCAGCTT CTCCTGGAGA GAAGGTGACT 60
ATGACTTGCA GGGCTTCTTC ATCTGTTTCT TACATCCACT GGTTCCAGCA GAAGCCTGGT 120
TCTTCACCTA AGCCTTGGAT CTACGCTACA TCTAACTTGG CATCTGGAGT GCCTGTGAGG 180
TTCTCTGGTT CTGGTTCAGG TACTTCTTAC TCTTTGACAA TCTCTAGGGT GGAGGCTGAG 240
GACGCTGCTA CTTACTACTG CCAGCAGTGG ACATCTAACC CTCCAACATT CGGAGGTGGT 300
ACTAAGTTGC AGATCAAC. 318

The CDRs of Rituximab are as follows. V_{H} comprises three CDRs: CDR-H1 (HCDR1), CDR-H2 (HCDR2) and CDR-H3 (HCDR3); V_{L} comprises three CDRs: CDR-L1 (LCDR1), CDR-L2 (LCDR2) and CDR-L3 (LCDR3).

Rituximab Heavy Chain
(SEQ ID NO: 9)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSA

| | | | Length |
|---|---|---|---|
| | | Residues of | (number of |
| | | SEQ ID | amino acid |
| Region | Sequence | NO: 9 | residues) |
| HFR1 | QVQLQQPGAELVKPGASVKMSCKASGYTFT | 1-30 | 30 |
| | (SEQ ID NO: 47) | | |
| CDR- | SYNMH (SEQ ID NO: 48) | 31-35 | 5 |
| H1 | | | |
| HFR2 | WVKQTPGRGLEWIG (SEQ ID NO: 49) | 36-49 | 14 |
| CDR- | AIYPGNGDTSYNQKFKG (SEQ ID NO: 50) | 50-66 | 17 |
| H2 | | | |
| HFR3 | KATLTADKSSSTAYMQLSSLTSEDSAVYYCAR | 67-98 | 32 |
| | (SEQ ID NO: 51) | | |
| CDR- | STYYGGDWYFNV (SEQ ID NO: 52) | 99-110 | 12 |
| H3 | | | |
| HFR4 | WGAGTTVTVSA (SEQ ID NO: 53) | 111-121 | 11 |

Rituximab Light Chain
(SEQ ID NO: 13)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIK

| | | | Length |
|---|---|---|---|
| | | Residues of | (number of |
| | | SEQ ID NO: | amino acid |
| Region | Sequence | 13 | residues) |
| LFR1 | QIVLSQSPAILSASPGEKVTMTC | 1-23 | 23 |
| | (SEQ ID NO: 54) | | |
| CDR-L1 | RASSSVSYIH (SEQ ID NO: 55) | 24-33 | 10 |
| LFR2 | WFQQKPGSSPKPWIY | 34-48 | 15 |
| | (SEQ ID NO: 56) | | |
| CDR-L2 | ATSNLAS (SEQ ID NO: 57) | 49-55 | 7 |
| LFR3 | GVPVRFSGSGSGTSYSLTISRVEAEDAATYYC | 56-7 | 32 |
| | (SEQ ID NO: 58) | | |
| CDR-L3 | QQWTSNPPT (SEQ ID NO: 59) | 88-96 | 9 |
| LFR4 | FGGGTKLEIK (SEQ ID NO: 60) | 97-106 | 10 |

Further, the amino acid sequence of the light chain variable region of the Obinutuzumab antibody used in the present disclosure is as follows:

(SEQ ID NO: 35)
DIVMTQTPLS LPVTPGEPAS ISCRSSKSLL HSNGITYLYW YLQKPGQSPQ LLIYQMSNLV 60
SGVPDRFSGS GSGTDFTLKI SRVEAEDVGV YYCAQNLELP YTFGGGTKVE IKRTV 115

The DNA sequence encoding the light chain variable region of the Obinutuzumab antibody is as follows:

(SEQ ID NO: 36)
gatatcgtga tgacccagac tccactctcc ctgcccgtca cccctggaga gcccgccagc 60
attagctgca ggtctagcaa gagcctcttg cacagcaatg gcatcactta tttgtattgg 120
tacctgcaaa agccagggca gtctccacag ctcctgattt atcaaatgtc caaccttgtc 180
tctggcgtcc ctgaccggtt ctccggctcc gggtcaggca ctgatttcac actgaaaatc 240
agcagggtgg aggctgagga tgttggagtt tattactgcg ctcagaatct agaacttcct 300
tacaccttcg gcggagggac caaggtggag atcaaacgta cggtg 345

The CDRs of Obinutuzumab are as follows. V_{H} comprises three CDRs: CDR-H1 (HCDR1), CDR-H2 (HCDR2) and CDR-H3 (HCDR3); V_{L} comprises three CDRs: CDR-L1 (LCDR1), CDR-L2 (LCDR2) and CDR-L3 (LCDR3).

Obinutuzumab Heavy Chain
(SEQ ID NO: 33)
QVQLVQSGAEVKKPGSSVKVSCKASGYAFSYSWINWVRQAPGQGLEWMGR
IFPGDGDTDYNGKFKGRVTITADKSTSTAYMELSSLRSEDTAVYYCARNV
FDGYWLVYWGQGTLVTVSS

| | | | Length |
|---|---|---|---|
| | | Residues of | (number of |
| | | SEQ ID | amino acid |
| Region | Sequence | NO: 33 | residues) |
| HFR1 | QVQLVQSGAEVKKPGSSVKVSCKASGYAFS | 1-30 | 30 |
| | (SEQ ID NO: 61) | | |
| CDR- | YSWIN (SEQ ID NO: 62) | 31-35 | 5 |
| H1 | | | |
| HFR2 | WVRQAPGQGLEWMG (SEQ ID NO: 63) | 36-49 | 14 |
| CDR- | RIFPGDGDTDYNGKFKG (SEQ ID NO: 64) | 50-66 | 17 |
| H2 | | | |
| HFR3 | RVTITADKSTSTAYMELSSLRSEDTAVYYCAR | 67-98 | 32 |
| | (SEQ ID NO: 65) | | |
| CDR- | NVFDGYWLVY (SEQ ID NO: 66) | 99-108 | 10 |
| H3 | | | |
| HFR4 | WGQGTLVTVSS (SEQ ID NO: 67) | 109-119 | 11 |

Obinutuzumab Light Chain
(SEQ ID NO: 35)
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKRTV

| | | | Length |
|---|---|---|---|
| | | Residues of | (number of |
| | | SEQ ID | amino acid |
| Region | Sequence | NO: 35 | residues) |
| LFR1 | DIVMTQTPLSLPVTPGEPASISC | 1-23 | 23 |
| | (SEQ ID NO: 68) | | |
| CDR- | RSSKSLLHSNGITYLY (SEQ ID NO: 69) | 24-39 | 16 |
| L1 | | | |
| LFR2 | WYLQKPGQSPQLLIY (SEQ ID NO: 70) | 40-54 | 15 |
| CDR- | QMSNLVS (SEQ ID NO: 71) | 55-61 | 7 |
| L2 | | | |
| LFR3 | GVPDRFSGSGSGTDFTLKISRVEAEDVGVYYC | 62-93 | 32 |
| | (SEQ ID NO: 72) | | |
| CDR- | AQNLELPYT (SEQ ID NO: 73) | 94-102 | 9 |
| L3 | | | |
| LFR4 | FGGGTKVEIKRTV (SEQ ID NO: 74) | 103-115 | 13 |

In one embodiment, the amino acid sequence of the linker between the heavy chain variable region and the light chain variable region is as follows:

(SEQ ID NO: 15)
GGGGSGGGGS GGGGS 15

Its coding DNA sequence is as follows:

(SEQ ID NO: 16)

| | |
|---|---|
| GGTGGCGGTG GCTCGGGCGG TGGTGGGTCG GGTGGCGGCG | 45 |
| GATCT | |

### Hinge Region and Transmembrane Domain (Region)

As for the hinge region and the transmembrane region (transmembrane domain), the CAR can be designed to comprise a transmembrane domain fused to the extracellular domain of the CAR. In one embodiment, a transmembrane domain that is naturally associated with one of the domains in the CAR is used. In some embodiments, transmembrane domains may be selected or modified by amino acid substitutions to avoid binding such domains to the transmembrane domain of the same or different surface membrane proteins, thereby minimizing the interaction with other members of the receptor complexes.

In one embodiment, the hinge region comprises the following amino acid sequence (IgG4 Hinge-CH2-CH3 hinge region):

(SEQ ID NO: 17)
ESKYGPPCPP CPAPEFLGGP SVFLFPPKPK DTLMISRTPE VTCVVVDVSQ EDPEVQFNWY 60
VDGVEVHNAK TKPREEQFNS TYRVVSVLTV LHQDWLNGKE YKCKVSNKGL PSSIEKTISK 120
AKGQPREPQV YTLPPSQEEM TKNQVSLTCL VKGFYPSDIA VEWESNGQPE NNYKTTPPVL 180
DSDGSFFLYS RLTVDKSRWQ EGNVFSCSVM HEALHNHYTQ KSLSLSLGK 229

Its coding DNA sequence is as follows:

| (SEQ ID NO: 18) | |
|---|---|
| GAGAGCAAGT ACGGACCGCC CTGCCCCCCT TGCCCTGCCC CCGAGTTCCT GGGCGGACCC | 60 |
| AGCGTGTTCC TGTTCCCCCC CAAGCCCAAG GACACCCTGA TGATCAGCCG GACCCCCGAG | 120 |
| GTGACCTGCG TGGTGGTGGA CGTGAGCCAG GAAGATCCCG AGGTCCAGTT CAATTGGTAC | 180 |
| GTGGACGGCG TGGAAGTGCA CAACGCCAAG ACCAAGCCCA GAGAGGAACA GTTCAACAGC | 240 |
| ACCTACCGGG TGGTCTCTGT GCTGACCGTG CTGCACCAGG ACTGGCTGAA CGGCAAAGAA | 300 |
| TACAAGTGCA AGGTGTCCAA CAAGGGCCTG CCCAGCAGCA TCGAAAAGAC CATCAGCAAG | 360 |
| GCCAAGGGCC AGCCTCGCGA GCCCCAGGTG TACACCCTGC CTCCCTCCCA GGAAGAGATG | 420 |
| ACCAAGAACC AGGTGTCCCT GACCTGCCTG GTGAAGGGCT TCTACCCCAG CGACATCGCC | 480 |
| GTGGAGTGGG AGAGCAACGG CCAGCCTGAG AACAACTACA AGACCACCCC TCCCGTGCTG | 540 |
| GACAGCGACG GCAGCTTCTT CCTGTACAGC CGGCTGACCG TGGACAAGAG CCGGTGGCAG | 600 |
| GAAGGCAACG TCTTTAGCTG CAGCGTGATG CACGAGGCCC TGCACAACCA CTACACCCAG | 660 |
| AAGAGCCTGA GCCTGTCCCT GGGCAAG; | 687 |

In one embodiment, the hinge region comprises the following amino acid sequence (IgG4 Hinge-CH2-CH3 (L235E, N297Q)):

| (SEQ ID NO: 19) | |
|---|---|
| ESKYGPPCPP CPAPEFEGGP SVFLFPPKPK DTLMISRTPE VTCVVVDVSQ EDPEVQFNWY | 60 |
| VDGVEVHNAK TKPREEQFQS TYRVVSVLTV LHQDWLNGKE YKCKVSNKGL PSSTEKTTSK | 120 |
| AKGQPREPQV YTLPPSQEEM TKNQVSLTCL VKGFYPSDIA VEWESNGQPE NNYKTTPPVL | 180 |
| DSDGSFFLYS RLTVDKSRWQ EGNVFSCSVM HEALHNHYTQ KSLSLSLGK | 229 |

Its coding DNA sequence is as follows:

| (SEQ ID NO: 20) | |
|---|---|
| GAGAGCAAGT ACGGACCGCC CTGCCCCCCT TGCCCTGCCC CCGAGTTCGA GGGCGGACCC | 60 |
| AGCGTGTTCC TGTTCCCCCC CAAGCCCAAG GACACCCTGA TGATCAGCCG GACCCCCGAG | 120 |
| GTGACCTGCG TGGTGGTGGA CGTGAGCCAG GAAGATCCCG AGGTCCAGTT CAATTGGTAC | 180 |
| GTGGACGGCG TGGAAGTGCA CAACGCCAAG ACCAAGCCCA GAGAGGAACA GTTCCAAAGC | 240 |
| ACCTACCGGG TGGTGTCTGT GCTGACCGTG CTGCACCAGG ACTGGCTGAA CGGCAAAGAA | 300 |
| TACAAGTGCA AGGTGTCCAA CAAGGGCCTG CCCAGCAGCA TCGAAAAGAC CATCAGCAAG | 360 |
| GCCAAGGGCC AGCCTCGCGA GCCCCAGGTG TACACCCTGC CTCCCTCCCA GGAAGAGATG | 420 |
| ACCAAGAACC AGGTGTCCCT GACCTGCCTG GTGAAGGGCT TCTACCCCAG CGACATCGCC | 480 |
| GTGGAGTGGG AGAGCAACGG CCAGCCTGAG AACAACTACA AGACCACCCC TCCCGTGCTG | 540 |
| GACAGCGACG GCAGCTTCTT CCTGTACAGC CGGCTGACCG TGGACAAGAG CCGGTGGCAG | 600 |
| GAAGGCAACG TCTTTAGCTG CAGCGTGATG CACGAGGCCC TGCACAACCA CTACACCCAG | 660 |
| AAGAGCCTGA GCCTGTCCCT GGGCAAG. | 687 |

In a preferred embodiment of the invention, the amino acid sequence of the transmembrane region derived from CD8 (CD8TM) is as follows:

| (SEQ ID NO: 21) | |
|---|---|
| IYIWAPLAGT CGVLLLSLVI TLYC | 24 |

The coding DNA sequence thereof is as follows:

| (SEQ ID NO: 22) | |
|---|---|
| ATCTACATCT GGGCGCCCTT GGCCGGGACT TGTGGGGTCC TTCTCCTGTC ACTGGTTATC | 60 |
| ACCCTTTACT GC | 72 |

In a preferred embodiment of the invention, the amino acid sequence of the transmembrane region derived from CD28 (CD28TM) is as follows:

| (SEQ ID NO: 37) | |
|---|---|
| FWVLVVVGGV LACYSLLVTV AFIIFWV | 27 |

The DNA sequence encoding the transmembrane region derived from CD28 (CD28TM) is as follows:

| (SEQ ID NO: 38) | |
|---|---|
| TTTTGGGTGC TGGTGGTGGT TGGTGGAGTC CTGGCTTGCT ATAGCTTGCT AGTAACAGTG | 60 |
| GCCTTTATTA TTTTCTGGGT G. | 81 |

### Intracellular Domain

The intracellular domain in the CAR may comprise the signaling domain of 4-1BB and the signaling domain of CD3ζ.

In one embodiment, the intracellular signaling domain of 4-1BB comprises the following amino acid sequence:

(SEQ ID NO: 23)

| | |
|---|---|
| KRGRKKLLYI FKQPFMRPVQ TTQEEDGCSC RFPEEEEGGC EL | 42 |

The coding DNA sequence thereof is as follows:

| (SEQ ID NO: 24) | |
|---|---|
| AAACGGGGCA GAAAGAAACT CCTGTATATA TTCAAACAAC CATTTATGAG ACCAGTACAA | 60 |
| ACTACTCAAG AGGAAGATGG CTGTAGCTGC CGATTTCCAG AAGAAGAAGA AGGAGGATGT | 120 |
| GAACTG | 126 |

In one embodiment, the intracellular signaling domain derived from CD28 comprises the following amino acid sequence:

(SEQ ID NO: 39)

| | |
|---|---|
| RSKRSRLLHS DYMNMTPRRP GPTRKHYQPY APPRDFAAYR S | 41 |

The coding DNA sequence thereof is as follows:

| (SEQ ID NO: 40) | |
|---|---|
| AGGAGTAAGA GGAGCAGGCT CCTGCACAGT GACTACATGA ACATGACTCC CCGCCGCCCC | 60 |
| GGGCCCACCC GCAAGCATTA CCAGCCCTAT GCCCCACCAC GCGACTTCGC AGCCTATCGC | 120 |
| TCC | 123 |

In one embodiment, the intracellular signaling domain of CD3ζ comprises the following amino acid sequence:

| (SEQ ID NO: 25) | |
|---|---|
| RVKFSRSADA PAYQQGQNQL YNELNLGRRE EYDVLDKRRG RDPEMGGKPQ RRKNPQEGLY | 60 |
| NELQKDKMAE AYSEIGMKGE RRRGKGHDGL YQGLSTATKD TYDALHMQAL PPR | 113 |

The coding DNA sequence thereof is as follows:

| (SEQ ID NO: 26) | |
|---|---|
| AGAGTGAAGT TCAGCAGGAG CGCAGACGCC CCCGCGTACA AGCAGGGGCA GAACCAGCTC | 60 |
| TATAACGAGC TCAATCTAGG ACGAAGAGAG GAGTACGATG TTTTGGACAA GAGACGTGGC | 120 |
| CGGGACCCTG AGATGGGGGG AAAGCCGAGA AGGAAGAACC CTCAGGAAGG CCTGTACAAT | 180 |
| GAACTGCAGA AAGATAAGAT GGCGGAGGCC TACAGTGAGA TTGGGATGAA AGGCGAGCGC | 240 |
| CGGAGGGGCA AGGGGCACGA TGGCCTTTAC CAGGGTCTCA GTACAGCCAC CAAGGACACC | 300 |
| TACGACGCCC TTCACATGCA GGCCCTGCCC CCTCGC | 336 |

### Vector

The present disclosure also provides a nucleic acid, a vector, or a DNA construct encoding the present CAR.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The present disclosure also provides vectors in which the DNA construct of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the advantage over vectors derived from onco-retroviruses such as murine leukemia viruses in that they can transduce non-proliferating cells, such as hepatocytes. They also have the advantage of low immunogenicity.

In certain embodiments, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The expression constructs of the present disclosure may also be used for nucleic acid immune and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the present disclosure provides a gene therapy vector.

The nucleic acid can be cloned into any suitable types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors,

Further, the expression vector may be provided to a cell in the form of a viral vector. Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of virus-based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters, inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to, a metallothionein promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In the case where a non-viral delivery system is utilized, genome editing technique may be exemplarily employed, for example CRISPR-Cas9, ZFN or TALEN.

In one embodiment, the vector is a lentiviral vector.

In certain embodiments, the DNA construct further comprises a signal peptide coding sequence. For example, the signal peptide sequence is ligated upstream of the nucleic acid sequence of antigen binding domain. In one embodiment, the signal peptide is a human CD8a signal peptide.

In one embodiment, the amino acid sequence of the signal peptide is as follows.

The amino acid sequence of CD8 leader sequence is:

| (SEQ ID NO: 27) | |
|---|---|
| MALPVTALLL PLALLLHAAR P | 21 |

The DNA sequence encoding CD8 leader sequence is:

| (SEQ ID NO: 28) | |
|---|---|
| ATGGCCTTAC CAGTGACCGC CTTGCTCCTG CCGCTGGCCT TGCTGCTCCA CGCCGCCAGG | 60 |
| CCG | 63 |

As used herein, the terms “CAR-T cell”, “CAR-T”, and “CART”, may be used interchangeably.

### Therapeutic Application

The present disclosure encompasses a cell (e.g., T cell) transduced with a lentiviral vector (LV) encoding the present CAR. The transduced T cell can elicit a CAR-mediated T-cell response.

Thus, the present disclosure also provides a method for stimulating a T cell-mediated immune response to a target cell population or tissue in a mammal comprising the step of administering to the mammal a T cell that expresses the present CAR.

In one embodiment, the present disclosure includes a cellular therapy where T cells are genetically modified to express the present CAR and the CAR-T cell is administered (e.g., infused) to a subject/recipient in need thereof. The administered (e.g., infused) cell is able to kill tumor cells in the recipient. Unlike antibody therapies, CAR-T cells are able to replicate in vivo resulting in long-term persistence that can lead to sustained tumor control.

In one embodiment, the CAR-T cells of the invention can undergo robust in vivo T cell expansion and can persist for an extended amount of time. In addition, the CAR mediated immune response may be part of an adoptive immunotherapy approach in which CAR-modified T cells induce an immune response specific to the antigen binding moiety in the CAR. For example, an anti-CD20 CAR-T cell elicits an immune response specific against cells expressing CD20.

Although the data disclosed herein specifically disclose lentiviral vector comprising anti-CD20 scFv, hinge and transmembrane domain, and 4-1BB and CD3ζ signaling domains, the disclosure should be construed to include any number of variations for each of the components of the construct as described elsewhere herein.

Diseases that may be treated using the present CAR, immune cells or pharmaceutical composition include CD20-positive tumors and diseases, e.g., caused by excessive B cells (such as autoimmune diseases, for example, lupus erythematosus, etc.). CD20 positive tumors may include CD20 positive non-solid tumors (such as hematological tumors, for example, leukemias and lymphomas) or solid tumors. Types of tumors or cancers to be treated with present CAR, immune cells or pharmaceutical composition include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, e.g., acute leukemias (such as acute lymphocytic leukemia, acute myelocytic leukemia, acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, mesothelioma, malignant lymphoma, pancreatic cancer and ovarian cancer.

The CAR-modified T cells of the disclosure may also serve as a type of vaccine for ex vivo immunization and/or in vivo therapy in a mammal. Preferably, the mammal is a human.

In certain embodiments, with respect to ex vivo immunization, at least one of the following occurs in vitro prior to administering the cell into a mammal: i) expansion of the cells, ii) introducing a nucleic acid encoding a CAR to the cells, and/or iii) cryopreservation of the cells.

Ex vivo procedures are well known in the art and are discussed more fully below. Briefly, cells are isolated from a mammal (preferably a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

In addition to using a cell-based vaccine in terms of ex vivo immunization, the present disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In certain embodiments, the CAR-modified T cells are used in the treatment of CCL. In certain embodiments, the cells of the invention are used in the treatment of patients at risk for developing CCL. Thus, the present disclosure provides methods for the treatment or prevention of CCL comprising administering to a subject in need thereof, a therapeutically effective amount of the CAR-modified T cells.

The CAR-modified T cells of the present disclosure may be administered either alone, or as a pharmaceutical composition in combination with diluents and/or with other components such as IL-2, IL-17 or other cytokines or cell populations. Briefly, pharmaceutical compositions of the present disclosure may comprise a cell population as described herein (e.g., immune cells expressing the CAR such as CAR-T cells), in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, or 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al, New Eng. J. of Med. 319: 1676, 1988). The optimal dosage and treatment regime for a particular patient can readily be determined by one skilled in the art of medicine by monitoring the patient for signs of disease and adjusting the treatment accordingly.

The administration of the compositions or cells may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions or cells of the present disclosure are administered to a patient by intradermal or subcutaneous injection. In another embodiment, the compositions or cells of the present disclosure are administered by i.v. injection. The compositions of T cells may be injected directly into a tumor, lymph node, or site of infection.

In certain embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to, treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the compositions or cells may be used in combination with chemotherapy, radiation, immunosuppressive agents, such as cyclosporin, azathioprine, methotrexate, mycophenolate, and FK506, antibodies, or other immunotherapeutic agents. In a further embodiment, the compositions or cells of the present disclosure are administered to a patient in conjunction with (e.g., before, simultaneously or following) bone marrow transplantation, or the use of chemotherapy agents such as, fludarabine, external-beam radiation therapy (XRT), cyclophosphamide For example, in one embodiment, subjects may undergo standard treatment with high dose chemotherapy followed by peripheral blood stem cell transplantation. In certain embodiments, following the transplant, subjects receive an infusion of the expanded immune cells of the present disclosure. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. In general, 1×10⁶ to 1×10¹⁰ of the modified T cells of the invention (e.g., CAR-T 20 cells) can be applied to patients by means of, for example, intravenous infusion each treatment or each course of treatment.

The advantages of the certain embodiments of the present disclosure include:
(1) As for the chimeric antigen receptor of the present disclosure, the extracellular antigen binding domain thereof is a specific anti-CD20 scFv. The CAR formed by binding the specific anti-CD20 scFv to a specific hinge region and an intracellular domain shows a great capability of killing tumor cells with low cytotoxicity and low side effects.
(2) The chimeric antigen receptor provided by the disclosure can achieve stable expression and membrane localization of CAR protein after T cells is infected by lentivirus carrying CAR gene.
(3) The CAR-modified T cell of the present disclosure has a longer survival time in vivo and strong anti-tumor efficacy. The optimized CAR with the IgG4 Hinge-CH2-CH3 linker region can avoid the binding of the Fc receptor and the subsequent ADCC effect (antibody-dependent cytotoxicity).

The term “about” may refer to a value or composition within an acceptable error range for a particular value or composition as determined by those skilled in the art, which will depend in part on how the value or composition is measured or determined. The term “about” in reference to a numeric value may refer to ±10% of the stated numeric value. In other words, the numeric value can be in a range of 90% of the stated value to 110% of the stated value.

The term “administering” refers to the physical introduction of a product of the disclosure into a subject using any one of various methods and delivery systems known to those skilled in the art, including, but not limited to, intravenous, intramuscular, subcutaneous, intraperitoneal, spinal or other parenteral administration, such as by injection or infusion.

The following examples of specific aspects for carrying out the present invention are offered for illustrative purposes only, and are not intended to limit the scope of the present invention in any way.

### EXAMPLE 1

### Construction of Lentiviral Expression Vector

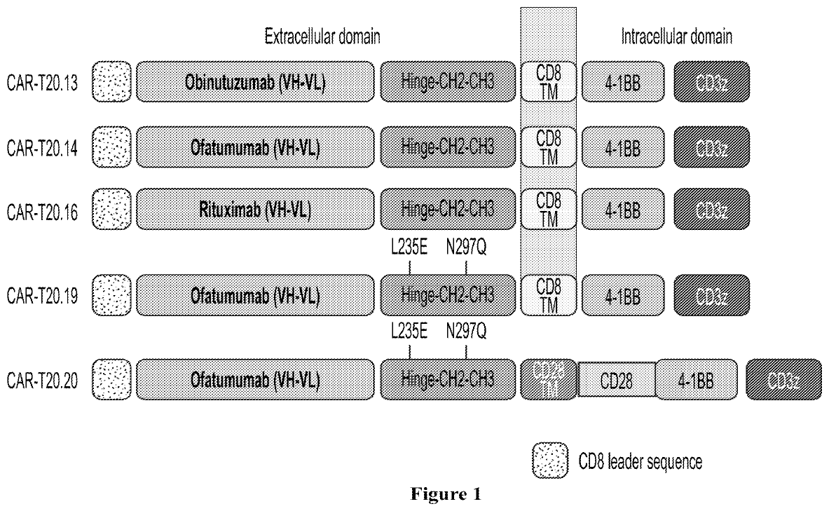

The full-length DNA synthesis and cloning construction of coding plasmids were conducted. Different anti-CD20 scFv coding sequences were used in each plasmid. The cloning vector was selected as pWPT lentiviral vector. The cloning sites were BamH I and Sal I sites. The specific sequence structure is shown in FIG. 1. The amino acid and nucleotide sequences of each element are as described above.

In the following examples, CAR-T20.13, CAR-T20.14, CAR-T20.16, CAR-T20.19, CAR-T20.20 with better effects are taken as examples.

### EXAMPLE 2

### Preparation of CAR-T Cell

(1) After taking venous blood from healthy subjects, mononuclear cells (PBMCs) were isolated by density gradient centrifugation.
(2) On day 0, PBMCs were cultured in GT-T551 cell culture medium containing 2% human albumin, and the final concentration of cells was adjusted to 2×10⁶ cells/mL. The cells were seeded in a cell culture flask previously coated with Retronectin (purchased from TAKARA) at a final concentration of 10 μg/mL and CD3 monoclonal antibody (OKT3) at a final concentration of 5 μg/mL. Recombinant human interleukin 2 (IL-2) was added to the culture medium at a final concentration of 1000 U/mL. The cells were cultured in an incubator with a saturated humidity and 5% CO₂ at 37° C.
(3) On day 2, fresh medium, concentrated and purified CAR20 lentivirus solution, protamine sulfate (12 μg/ml), and IL-2 (at a final concentration of 1000 U/mL) were added. After 12 hours of infection in a 5% CO₂ incubator at 37° C., the culture medium was discarded, fresh medium was added, and cultivation was continued in a 5% CO₂ incubator at 37° C.
(4) Starting from day 6, CART20 cells can be taken for the corresponding activity assay.

In the present disclosure, the preparation process of CAR-modified T cell targeting CD20 antigen was improved, and GT-551 serum-free medium supplemented with 2% human albumin was selected to culture lymphocytes in vitro.

### EXAMPLE 3

### Detection of the Integration Rate of the CAR Gene in the T Cell Genome and the Expression Level of the Encoded Protein Thereof on the Membrane Surface

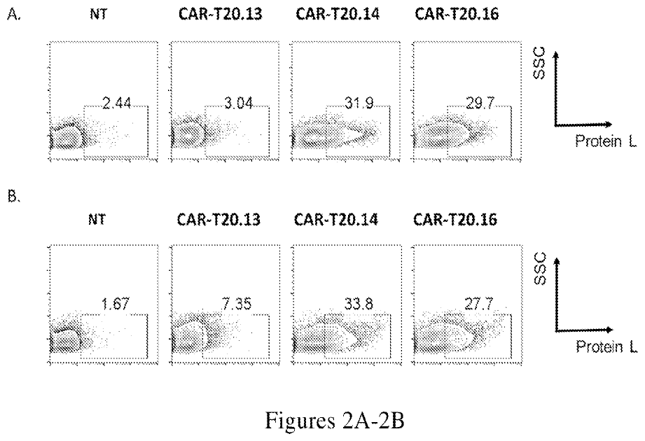

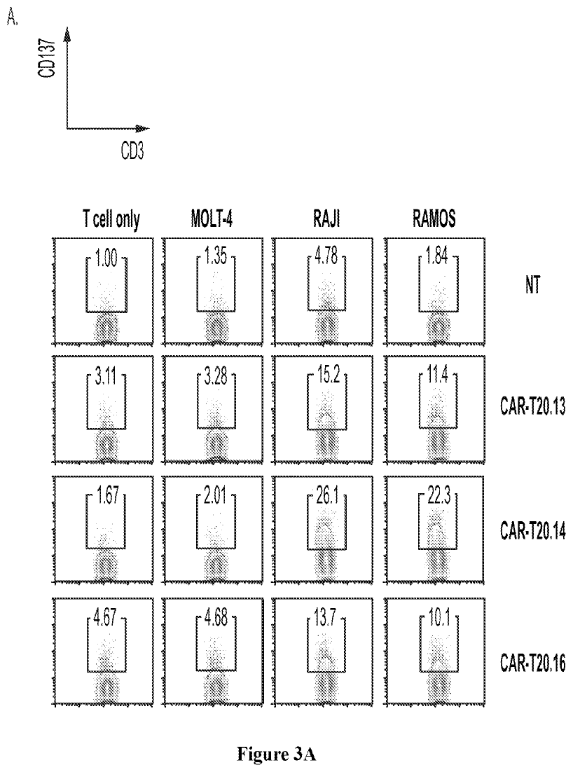

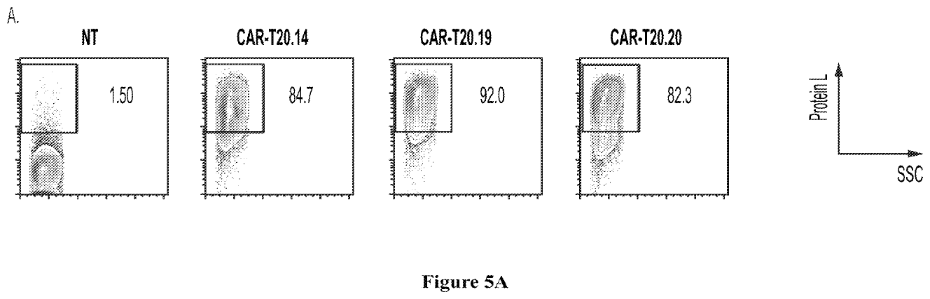

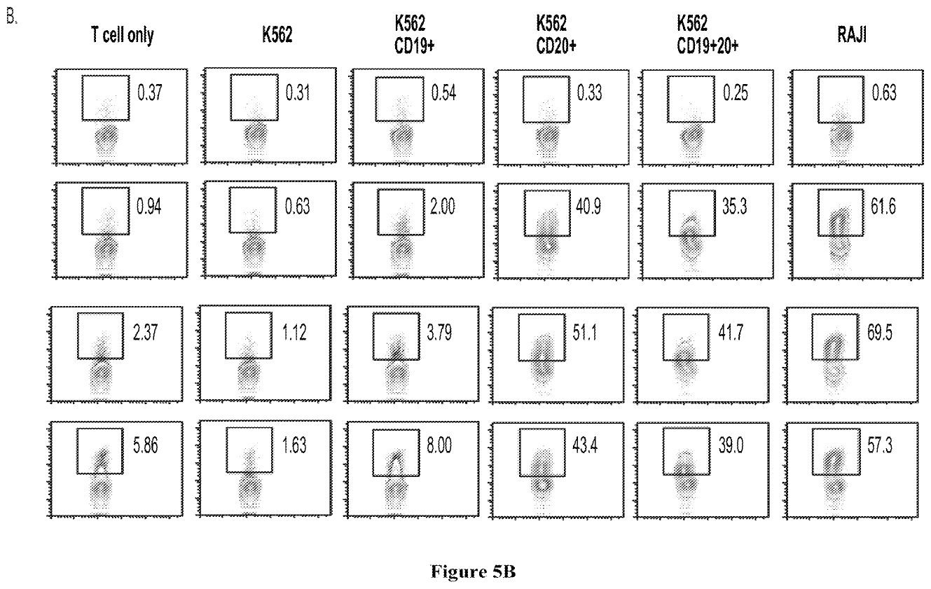

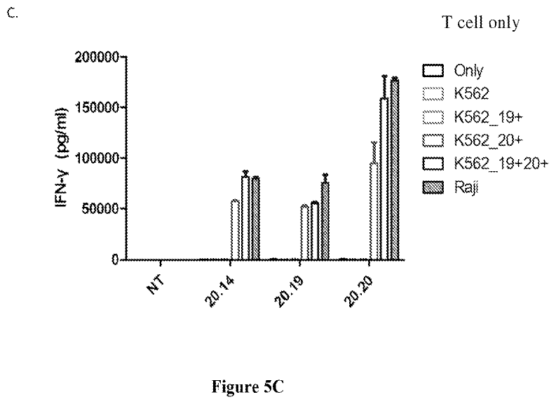

0.5×10⁶ of CART-20 cell samples cultured on day 7 (FIG. 2A and FIG. 5A) and day 11 (FIG. 2B) in Example 2 were taken, respectively. The expression level of CAR20 protein on the surface of T cell membrane was analyzed by flow cytometry after Protein L staining. The results showed that, except for CAR-T20.13, all of the CAR structures designed in this study can detect the chimeric antigen receptor localization on the cell membrane surface of the corresponding modified T cells using Protein L.

### EXAMPLE 4

### Detection of the In Vitro Activation Ability of CAR-T20s

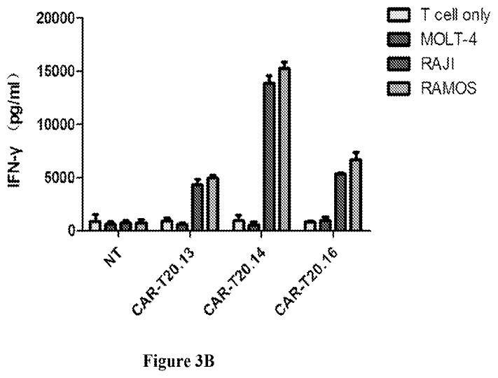

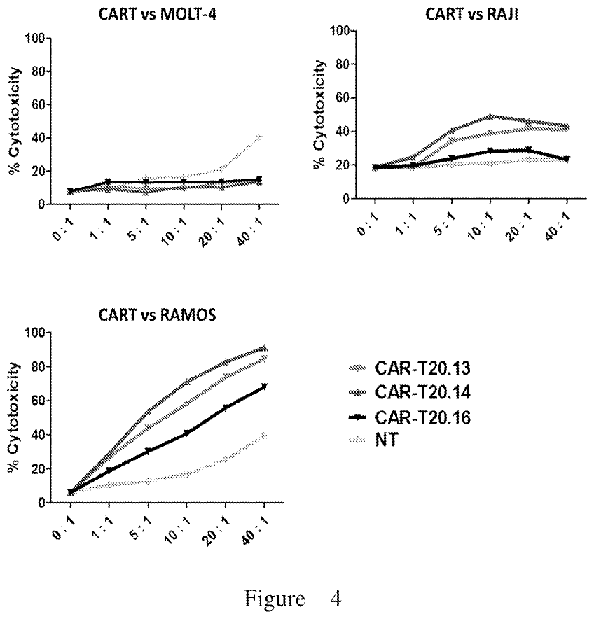

The deCAR-T20 cells cultured on day 6 in Example 2 were co-cultured with target cells. Then the up-regulated level of CD137 and the secretion level of IFNγ in the culture supernatant were examined 1×10⁵ of CART-20 cells (cultured on day 6) were cultured respectively with CD20-positive RAJI and RAMOS tumor cell lines, and CD20-negative MOLT-4 tumor cell line, or without tumor cells, in 200 μl GT-551 medium for 18 h in a ratio of 1:1. Then the expression level of CD137 on the surface of T cell membrane was detected by flow cytometry (FIG. 3A) and the secretion level of IFNγ in the culture supernatant was detected by ELISA (FIG. 3B).

From the results in FIGS. 3A-3B, we could concluded that the CAR based on Obinutuzumab also achieved expression and membrane surface localization in the corresponding modified cells, but the CAR structure based on the Ofatumumab sequence showed better in vitro activation ability and specificity targeting antigen when compared with the CAR constructed based on Obinutuzumab and Rituximab

### EXAMPLE 5

### Detection of the CAR-T20s Cells Induced Early Apoptosis Activity of Tumor Cells

CART-20.13, CART-20.14 and CAR-T20.16 cells (cultured on day 11) from Example 2 were co-cultured respectively with 1×10⁴ of CFSE-labeled CD20-negative (MOLT-4) or CD20-positive (RAJI, RAMOS) tumor cell lines in 200 μl GT-551 medium for 4 h. Then the cell pellet was collected by centrifugation. The cells were washed twice with PBS and stained for 30 min with Annexin V-APC dye in a ratio of 1:50 in 100 μl of dyeing solution. After washing with PBSonce, the proportion of Annexin V positive cells in CFSE positive cells was analyzed on a flow cytometry.

The results in FIG. 4 show that the CAR structure based on the Ofatumumab sequence shows better ability to induce early apoptosis of CD20 target cells in vitro when compared with the CAR constructed based on Obinutuzumab and Rituximab.

### EXAMPLE 6

### Identification of the In Vitro Activation Ability of the Third-Generation Chimeric Antigen Receptor and the Chimeric Antigen Receptor With Mutation in Hinge Region

(1) Under the condition that the transfection rate was basically equal (FIG. 5A), the CAR-T20s cells (prepared by the method of Example 2) cultured on the day 7 were cultured respectively with K562, K562 stable transfected cells of CD19 single positive, CD20 single positive, CD19 and CD20 double positive, and RAH target cell (each taking 1×10⁵ cells) in 200 μl GT-551 medium for 18 h in a ratio of 1:1. Then the up-regulated level of CD137 (FIG. 5B) and the secretion level of IFNγ in the culture supernatant (FIG. 5C) were detected.
(2) The results shown in FIG. 5 indicate that the in vitro activation ability (CD137 and IFNg) of the chimeric antigen receptor CAR-T20.14 and CAR-T20.19 (having a mutation in the hinge region) is substantially equivalent, in the case of substantially identical infection efficiency. The third generation CAR structure CAR-T20.20 shows better in vitro activation capacity (CD137 and IFNγ) than the second-generation CAR-T20.14 and CAR-T20.19.

### EXAMPLE 7

### Detection of the Ability of CAR-T20 Cells to Scavenge CD20 Positive Cells In Vivo

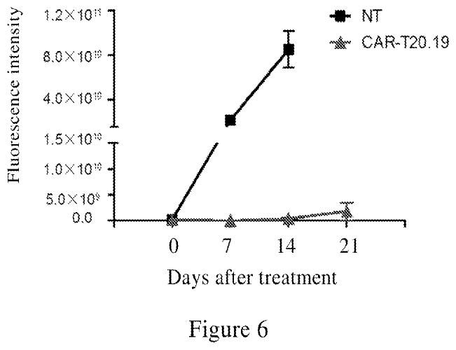

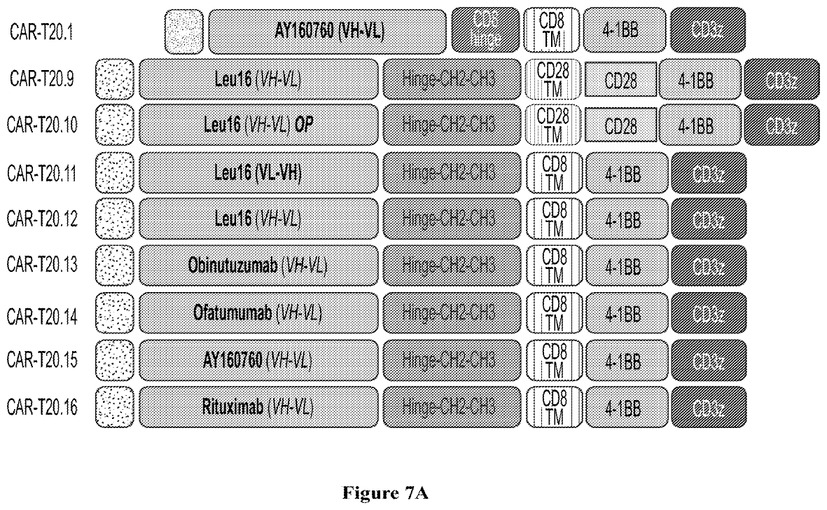

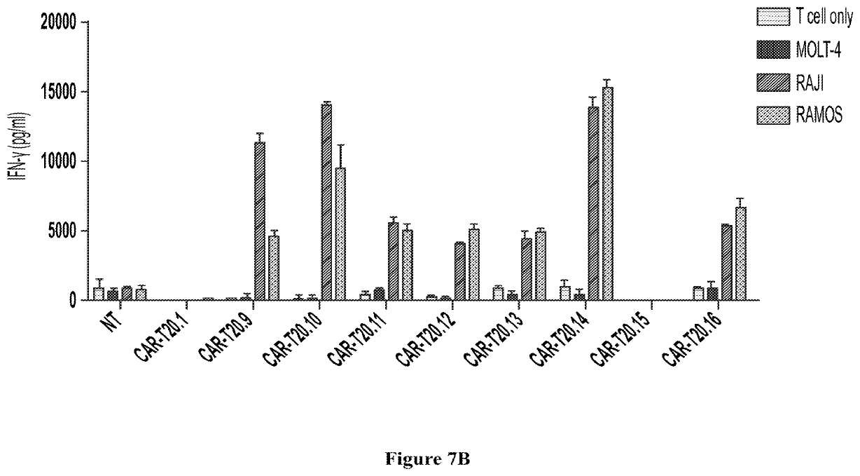

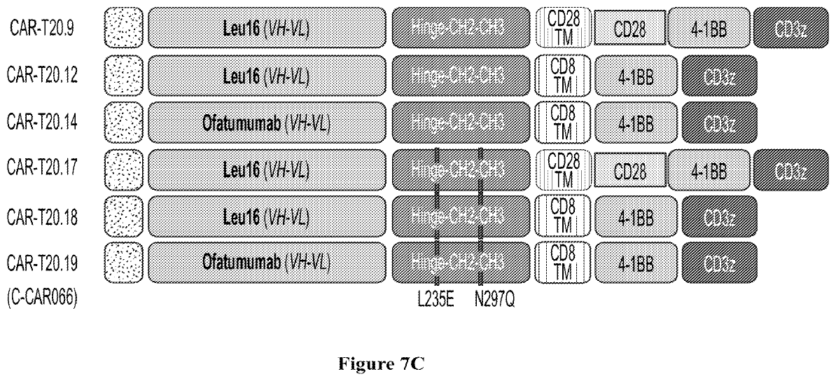

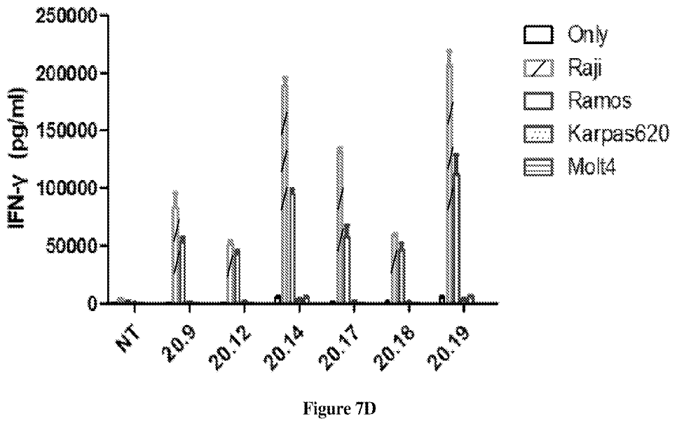

(1) Raji-Luc cells expressing luciferase were injected into NCG mice (5×10⁵/mouse) through the tail vein. One week after the inoculation, the in vivo expansion of the tumor cells was observed by in vivo imaging and recorded as Day 0. NT and CAR-T20.19 cells were injected into Day 0 mice (5×10⁶/mouse) through the tail vein. On Day0, Day7, Day14, Day21, the expansion of tumor cells in mice was observed by in vivo imaging and analyzed based on changes in fluorescence intensity and body weight changes of mice.
(2) The results shown in FIG. 6 indicate that CAR-T20.19 can effectively inhibit the in vivo expansion of CD20-positive tumor cells.

### EXAMPLE 8

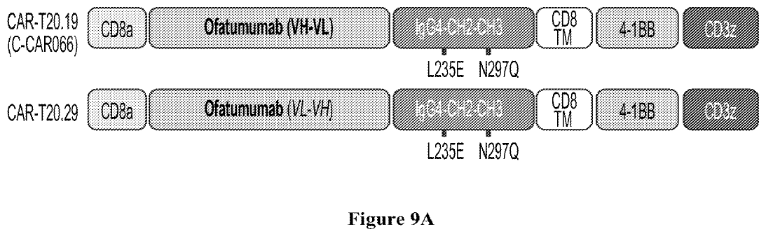

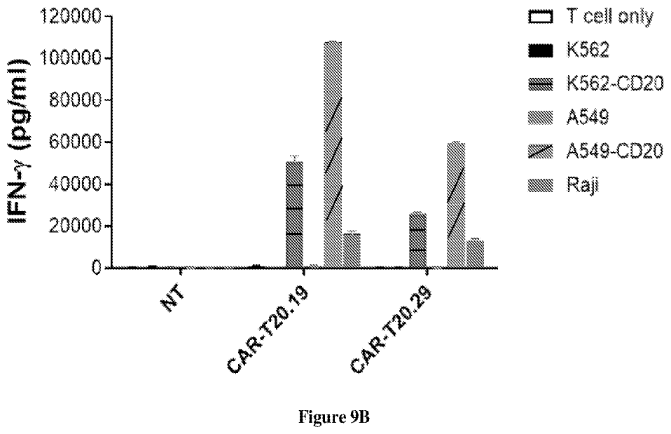

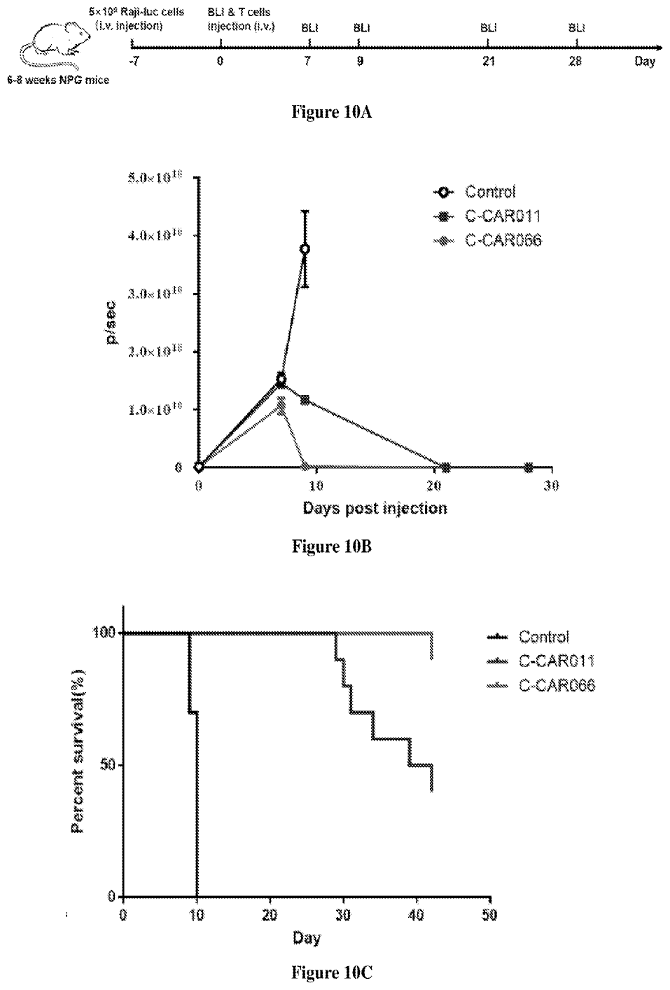

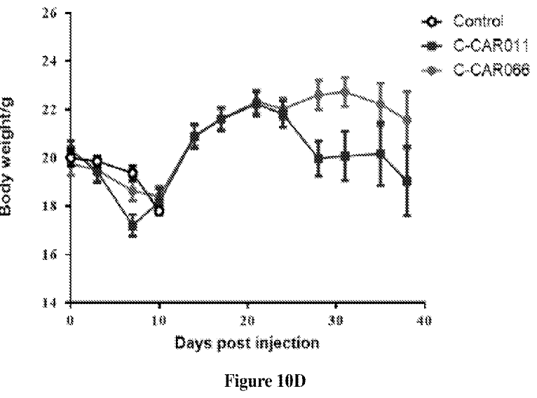

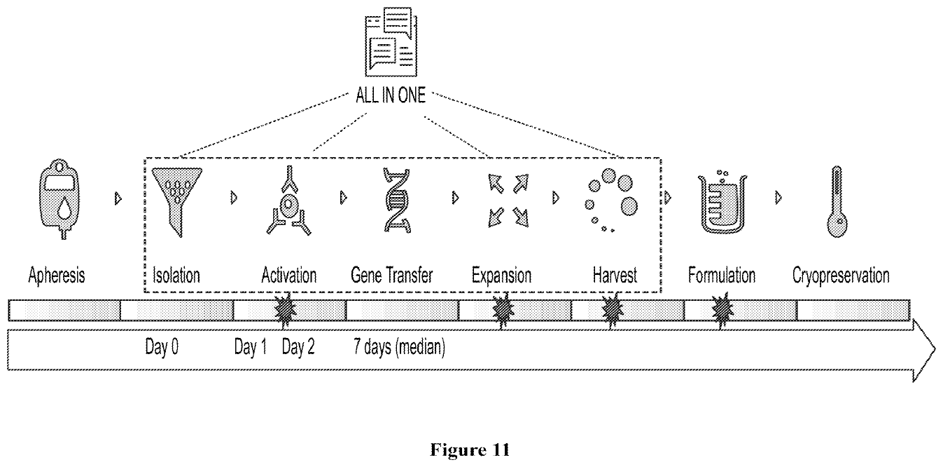

We prepared two anti-CD20 CARs having the same V_{H} (SEQ ID NO: 7) and V_{L} (SEQ ID NO: 11) but in different orders: CAR-T20.19 (SEQ ID No. 5) has V_{H}-V_{L} (i.e., V_{H} is located at the N-terminus of V_{L}), while CAR-T20.29 has V_{L}-V_{H} (FIG. 9A).

As shown in FIG. 9B, CAR-T20.19 (V_{H}-V_{L}) demonstrated significantly higher in vitro activities (e.g., inducing interferon-γ or IFN-γ release) against the CD20-positive cells than CAR-T20.29 (V_{L}-V_{H}). Specifically, CAR-T20.19 (V_{H}-V_{L}) showed 190%, 80%, and 38% greater activities compared to CAR-T20.29 (V_{L}-V_{H}) for the CD20-positive cell lines K562-CD20, A549-CD20, and Raji, respectively.

Thus, the order of V_{H} and V_{L} directly impacts the function of the CAR T cells. CARs having the V_{H}-V_{L} structure (e.g., CAR-T20.19) demonstrated significantly higher in vitro activities than CARs having the reversed V_{L}-V_{H} structure (e.g., CAR-T20.29).

### EXAMPLE 9

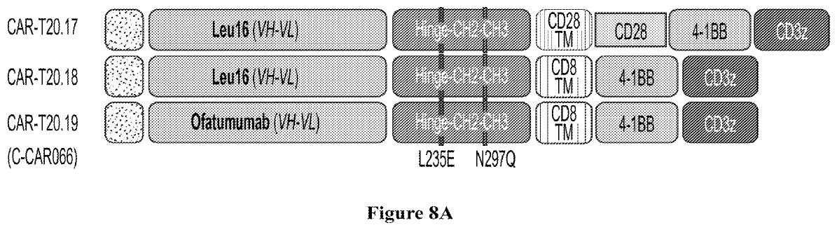

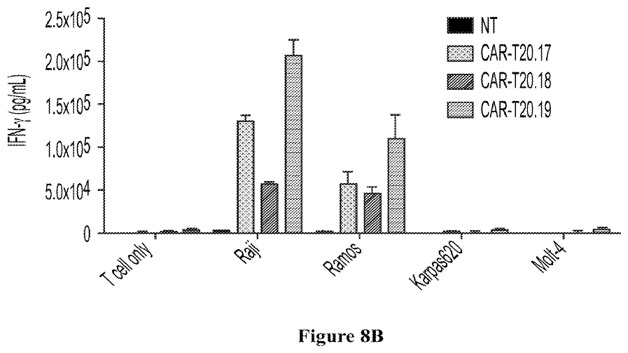

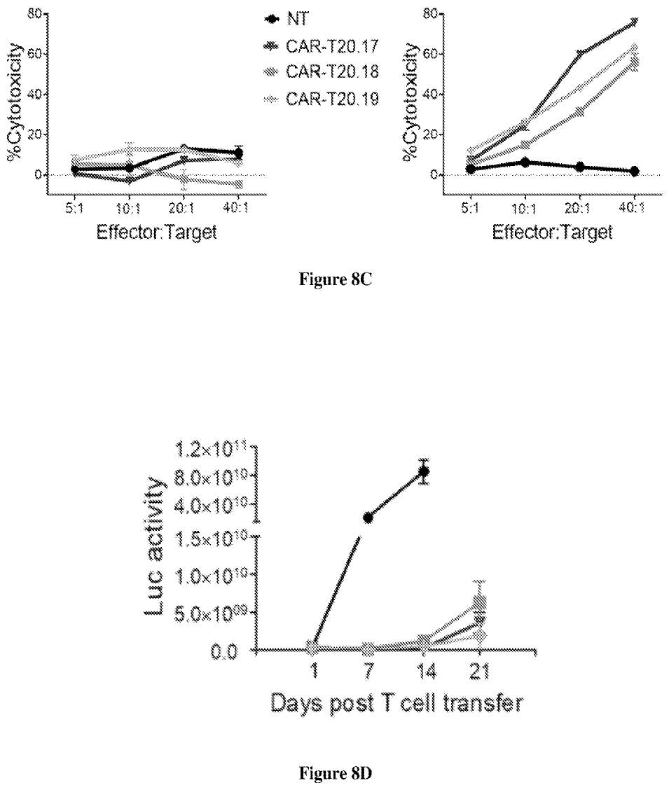

Our studies demonstrated that CAR-T20.19 was considerably more cytotoxic towards tumor cells both in vitro and in vivo, compared to CAR-T cells based on another anti-CD20 antibody, Leu16. FIGS. 8B-8C show CAR-T20.19 induced higher levels of IFN-γ release and greater cell killing in CD20-positive tumor cells, including RAJI and RAMOS, compared to CART20-Leu.

For in vivo studies, NSG mice were xenografted with Raji-Luc cells which are human Burkitt's lymphoma Raji cells expressing firefly luciferase as a reporter. Different CAR-T cells or negative control were then administered to the mice. The fluorescence intensity of the animals xenografted with Raji-Luc were assayed after treatment, which reflected the proliferation of tumor cells in the animals. FIG. 8D shows that the fluorescence intensities in the mice administrated with CART20-OF(2nd) (CAR-T20.19) T cells were markedly lower than mice administered with CART20-LEU (2nd) or CART20-LEU (3rd). The results suggest that CAR-T20.19 had higher in vivo anti-tumor efficacy than the other CARs.

The above in vitro and in vivo data prove that, compared with CAR-T cells with other scFv sequences, CART20-OF(2nd) (CAR-T20.19) possesses superior anti-tumor efficacy both in vitro and in vivo.

### EXAMPLE 10

B-cell lymphomas can be stratified into Hodgkin lymphoma (˜10% of all cases) and non-Hodgkin lymphoma (NHL; ˜90% of all cases), both of which comprise many subtypes. For relapsed and refractory NHL, the response rates to conventional salvage chemotherapy are approximately 40-50%. NHL subtypes include indolent forms, such as follicular lymphoma (FL), and aggressive forms, such as diffuse large B-cell lymphoma (DLBCL). Standard therapies for lymphoma include combination immunotherapy/chemotherapy, radiation therapy, and hematopoietic stem cell transplant (HSCT). NHL is associated with high mortality and a poor prognosis. The prognosis for patients with DLBCL is even grimmer, where the overall survival is 6.3 months from the last treatment failure. A study reported 43% overall response rate (ORR) in patients with DLBCL and 71% ORR in those with FL at 6 months after anti-CD19 CAR-T cell infusion. See, Lulla et al., The Use of Chimeric Antigen Receptor T Cells in Patients with Non-Hodgkin Lymphoma, Clinical Advances in Hematology & Oncology, 2018, 16(5): 375-386.

We conducted a clinical trial of CAR-T20.19 (also termed “C-CAR066”) in treating relapsed/refractory DLBCL (R/R DLBCL) in patients who were released from the anti-CD19 CAR-T treatment and had one or more relapses prior to our CAR-T20.19 clinical trial. These patients had very poor clinical outcome.

Specifically, ten (10) patients were enrolled. The patients' baseline demographics and clinical characteristics prior to the start of our anti-CD20 CAR treatment are shown in Table 1.

TABLE 1
Summary of demographic and baseline clinical
characteristics of the patients

| Characteristic | N = 10 |
|---|---|
| Median age, yrs (range) | 55.5 (41-67) |
| Age ≥ 65, n (%) | 2 (20.0) |
| Male, n (%) | 5 (50.0) |
| NHL Subtype, n (%) | |
| DLBCL, NOS | 8 (80.0) |
| tFL | 2 (20.0) |
| ECOG PS, n (%) | |
| 0 | 1 (10.0) |
| 1 | 9 (90.0) |
| IPI score 3-5, n (%) | 6 (60.0) |
| Ann Anbor stage III/IV, n (%) | 9 (90.0) |
| Double-expressor lymphoma, n (%) | 4 (40.0) |
| Median number of prior lines of therapy, n (range) | 5 (2-6) |
| 2, n (%) | 1 (10.0) |
| 4, n (%) | 3 (30.0) |
| 5, n (%) | 4 (40.0) |
| 6, n (%) | 2 (20.0) |
| Prior ASCT, n (%) | 2 (20.0) |
| Prior BTK inhibitor, n (%) | 2 (20.0) |
| Prior Lenalidomide, n (%) | 6 (60.0) |
| Best response to prior CAR-T therapy, n (%) | |
| CR | 2 (20.0) |
| PR | 8 (80.0) |
| Duration of response of prior CAR-T therapy, m (range) | 2.1 (0.7-12.6) |
| Received bridging therapy, n (%) | 4 (40.0) |

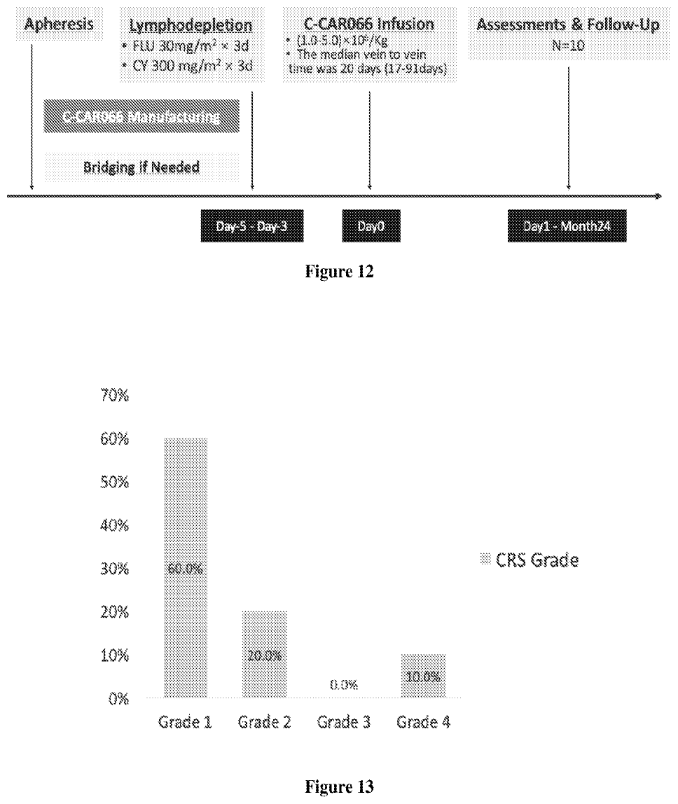

The clinical protocol, as well as the key inclusion criteria, is shown in FIG. 12. Specifically, the patients were screened 21 days before the treatment (−21 d). Qualified subjects were enrolled, and peripheral blood leukocytes collected. The collected peripheral blood leukocytes were used to produce the CAR-T cells (CAR-T20.19). The CAR-T cells were then frozen and stored until use. For CAR-T treatment, the CAR-T cells were thawed, and administration completed within 30-45 minutes.

At −5, −4 and −3 days before the CAR-T infusion, the patients received lymphodepletion pretreatment, including fludarabine (30 mg/m²/d, intravenous, once per day for three days), and cyclophosphamide (300 mg/m²/d, intravenous, once per day for three days).

Approximately 72 hours after lymphodepletion, the patients were administered 2.0×10⁶, 3.0×10⁶ or 4.8×10⁶ CAR-T cells/kg on day 0 as a single infusion. Follow-ups with the patients were carried out from day 1 to month 24 (e.g., day 4, day 7, day 10, week 2, week 3, week 4, etc.) after the infusion. The first clinical response assessment was at week 4 after the CAR-T infusion.

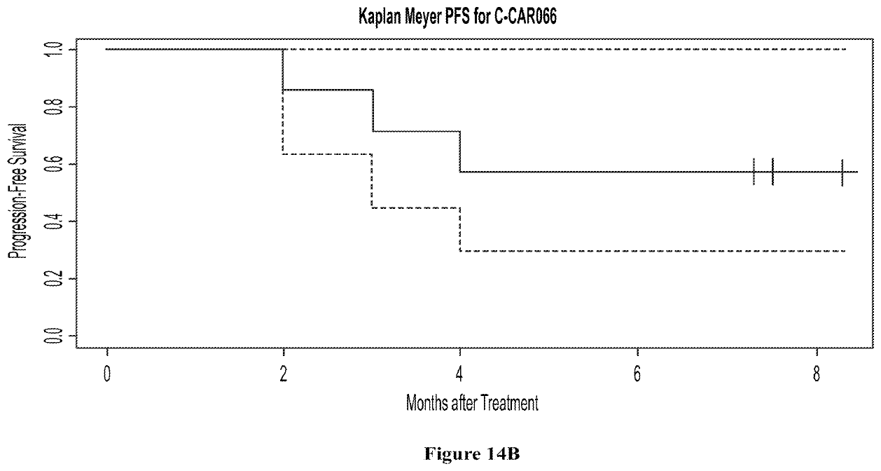

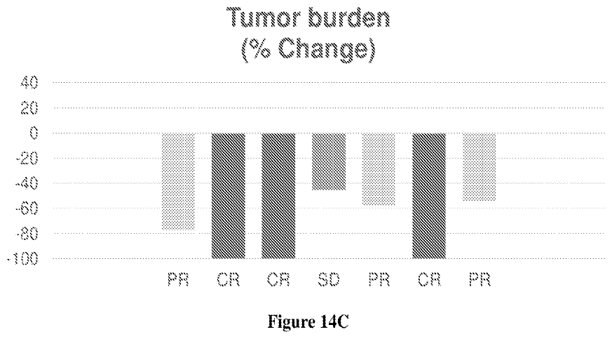

For our clinical trial of CAR-T20.19, the Kaplan Meier progression-free survival (PFS) estimates include a 6-month PFS of 57.1%, with 95% confidence intervals (CIs) of ˜30%-100%. See Table 2 and FIG. 14B.

TABLE 2

| | | | | std. | lower | upper |
|---|---|---|---|---|---|---|
| Time (month) | n.risk | n.event | survival | err. | 95% CI | 95% CI |
| 0 | 7 | 0 | 1.000 | 0 | 1.000 | 1 |
| 1 | 7 | 0 | 1.000 | 0 | 1.000 | 1 |
| 2 | 7 | 1 | 0.857 | 0.132 | 0.633 | 1 |
| 3 | 6 | 1 | 0.714 | 0.171 | 0.447 | 1 |
| 4 | 5 | 1 | 0.571 | 0.187 | 0.301 | 1 |
| 5 | 4 | 0 | 0.571 | 0.187 | 0.301 | 1 |
| 6 | 4 | 0 | 0.571 | 0.187 | 0.301 | 1 |
| 7 | 4 | 0 | 0.571 | 0.187 | 0.301 | 1 |
| 8 | 1 | 0 | 0.571 | 0.187 | 0.301 | 1 |

The tumor burden in the patients decreased significantly (FIG. 14C).

The patients' adverse reactions (treatment-emergent adverse events, TEAE) were recorded (Table 3 and Table 4). There was only 1 (10.0%) grade≥3 cytokine release syndrome (CRS). No neurotoxicity was observed in the patients. Cytopenia, such as neutropenia and thrombocytopenia, was mostly related to the fludarabine/cyclophosphamide (Cy/Flu) lymphodepletion. The cytopenia was also reversible. These demonstrated that our anti-CD20 CAR had an excellent safety profile.

TABLE 3

| | All Grades | Grade ≥ 3 |
|---|---|---|
| AEs, n (%) | (n = 10) | (n = 10) |
| CRS (Cytokine release | 9 (90.0%) | 1 (10.0%) |
| syndrome)¹ | | |
| ICANS | 0 (0) | 0 (0) |
| Neutropenia | 10 (100%) | 8 (80.0%) |
| Anemia | 10 (100%) | 5 (50.0%) |
| Thrombocytopenia | 7 (70.0%) | 3 (30.0%) |
| Infection | 7 (70.0%) | 1 (10.0%) |

¹CRS: uniformly graded according to the ASTCT Guidelines. See, Lee, Biol Blood Marrow Transplant, 2019, 25:625.

TABLE 4

| CRS | N = 10 |
|---|---|
| CRS, n (%) | 9 (90.0%) |
| Median days to onset, d (range) | 2 (1-9) |
| Median days to resolution, d (range) | 4 (2-17) |
| Treated with Tocilizumab alone, n (%) | 0 (0) |
| Treated with steroids alone, n (%) | 0 (0) |
| Treated with Tocilizumab and steroids, n (%) | 1 (10.0) |

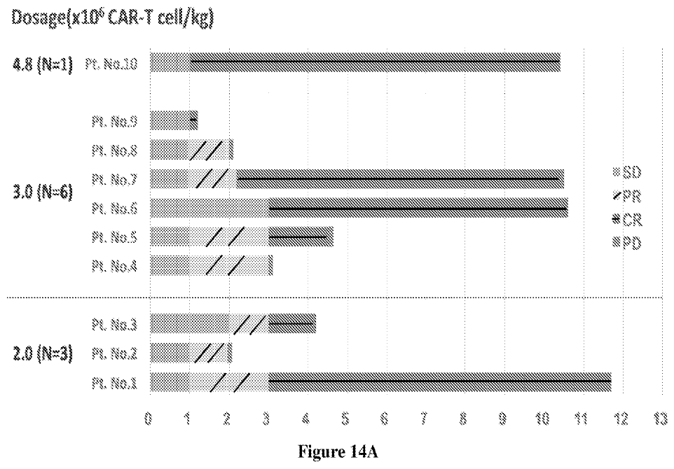

8 out of 10 patients had grade 1-2 CRS, while 1 out of 10 patients had grade 4 CRS. This patient presented with high fever, hypotension and hypoxia on day 6 and resolved on day 10. The patient was treated with tocilizumab and steroids, and with non-invasive ventilation support. The patient was not admitted to the ICU. There had not been ICANS events. See also, FIG. 13. Cytopenias mostly related to Cy/Flu lymphodepletion which were reversible.

As shown in FIG. 14A and Table 5, an overall response rate (ORR, including CR and PR) of our anti-CD20 CAR-T trial is 100%. The best response included 7 CRs (70.0%) and 3 PRs.

TABLE 5

| Response* | N = 10 |
|---|---|
| ORR, n (%) | 10 (100) |
| CR rate | 7 (70.0) |
| PR rate | 3 (30.0) |
| Median time to response, m (range) | 1.0 (0.9-2.7) |
| Median duration of response, m (range) | NR (1.0-NR) |
| Median time to CR, m (range) | 2.7 (0.9-2.9) |
| Median duration of CR, m (range) | NR (1.5-NR) |
| Median follow-up, m (range) | 4.2 (1.2-11.7) |

*Assessed by investigators.
NR: not reached.

As shown in FIG. 14A, ORR rate was 100% and CR rate was 70%. The median time to first response was 1 month (range, 0.9-2.7). The median time to CR as 2.7 months (range, 0.9-2.9). Median follow-up was 4.2 months (range is 1.2-11.7). Median DOR has not been reached. 4 patients remained in CR after 10 months.

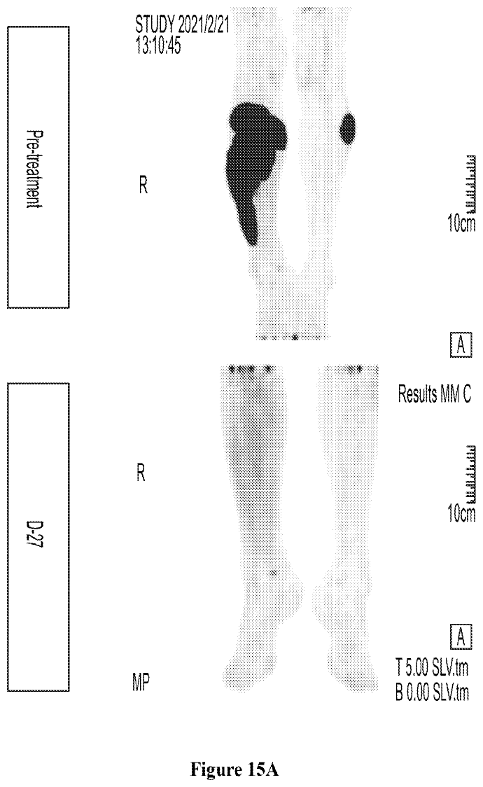

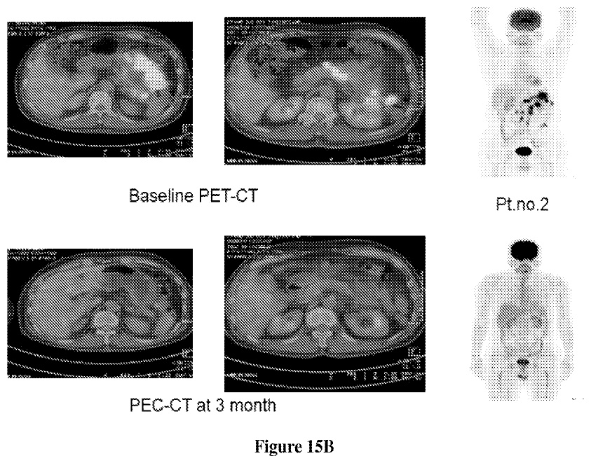

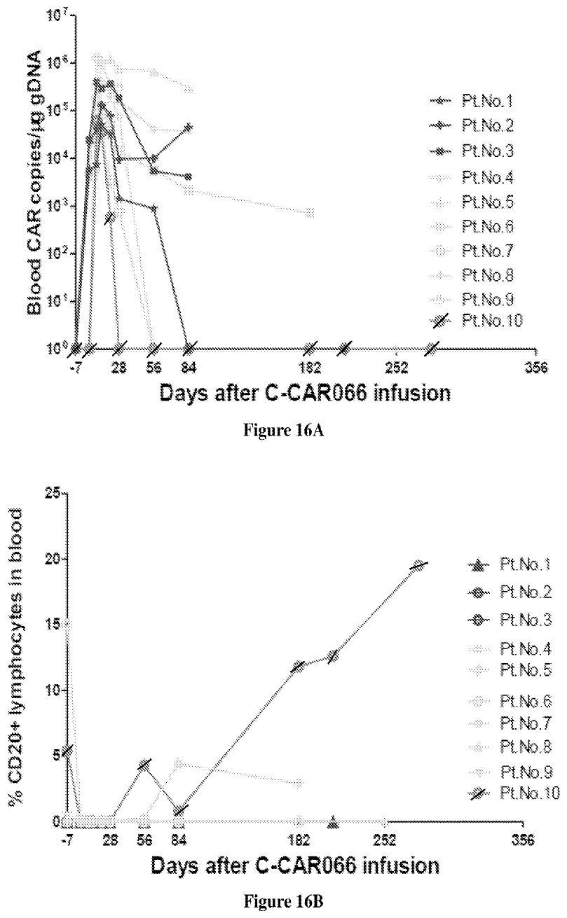

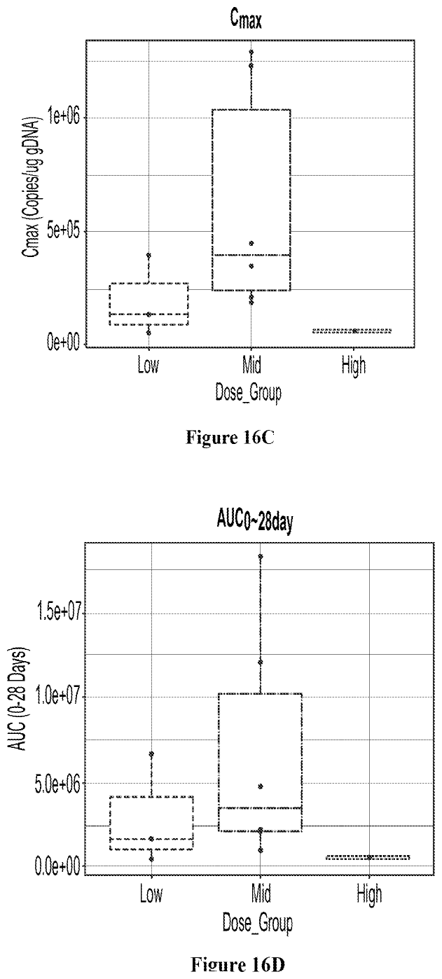

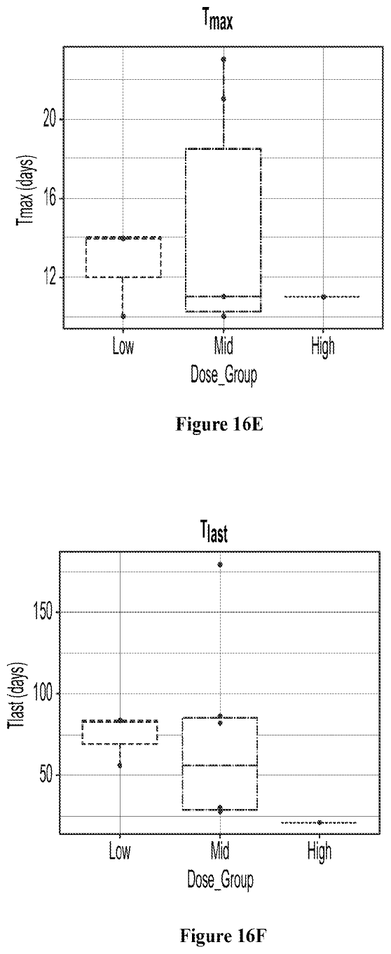

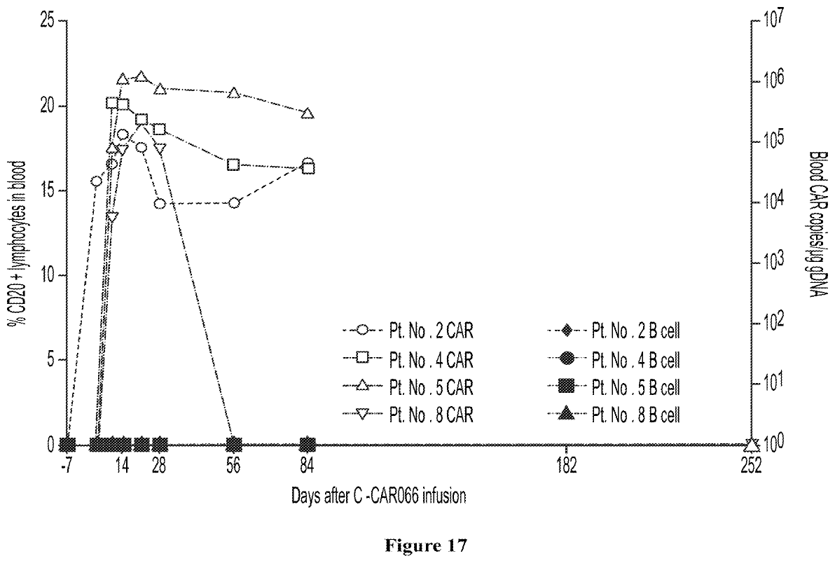

The time course of the CAR copies in the blood of the patients is shown in FIG. 16A. Thus, the CAR levels were maintained in the blood after administration.

The PET-CT images of the cancer lesions for one patient, patient No. 2, are shown in FIG. 15B. It clear shows that the tumor lesions decreased significantly in size three months after the anti-CD20 CAR T treatment.

CD19/CD20 expression tested in tumor tissues by IHC is shown in Table 6.

TABLE 6

| | Before C-CAR066 | Relapsed after C-CAR066 |
|---|---|---|
| Patient | treatment | treatment |
| Patient No. 2 | CD19(+)CD20(+) | CD19(+)CD20(+) |
| Patient No. 4 | CD19(dim)CD20(+) | CD19(−)CD20(−) |
| Patient No. 5 | CD19(+)CD20(+) | CD19(−)CD20(−) |
| Patient No. 8 | CD19(UK)CD20(+) | CD19(+)CD20(+) |

The aggressive forms of lymphomas, such as DLBCL, are less susceptible to T cell-mediated immune effects than indolent lymphomas. Thus, the fact that CAR-T20.19 achieved 100% overall response rate (ORR) and 70.0% complete response rate (CR) in treating R/R DLBCL, after post-anti-CD19 CAR-T treatment relapses, with a single administration is notable.

To summarize, CAR-T20.19 offered superior therapeutic efficacy in a clinical trial, with high response rates (100% ORR and 70.0% CR) in treating relapsed/refractory non-Hodgkin lymphoma (R/R NHL) and a favorable safety profile. The remarkable 100% ORR and 70.0% CR were achieved after only a single administration of the anti-CD20 CAR T cells.

Preclinical studies suggest that C-CAR066 has optimal structure and superior anti-tumor activity compared to anti-CD20 CAR-Ts derived from scFvs of Leu16, Rituximab, and Obinutuzumab and anti-CD19 CAR-T. In the clinical study, C-CAR066 shows a favorable safety profile and very promising efficacy in patients with r/r NHL following CD19 CAR-T therapy (with a median DOR of 2.1 months) compared to CD20/CD3 bispecific antibody.

### EXAMPLE 11

In one case study, a 67-year-old male with double-expressor DLBCL was diagnosed in May 2019. The patient had 4 prior lines of therapy, including anti-CD19 CAR-T treatment. The patient had right and left calve lesions. The patient's bulky disease was 25.9*6.3*10.1 cm in the right leg at baseline. The prior anti-CD19 CAR-T treatment had a best response of PR and duration of response of 1.2 months. The C-CAR066 treatment included 3.0×10⁶/kg dosage, grade 2 CRS (onset on day 2, resolved on day 11), no neurotoxicity. CR was achieved by day 27 (FIG. 15A).

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

1. A chimeric antigen receptor (CAR), comprising: an anti-CD20 antigen-binding region which comprises a heavy chain variable region (V_{H}) and a light chain variable region (V_{L}), V_{H} comprising three CDRs, HCDR1, HCDR2 and HCDR3, V_{L} comprising three complementarity determining regions (CDRs), LCDR1, LCDR2 and LCDR3,
(a) wherein HCDR1, HCDR2 and HCDR3 have amino acid sequences about 80% to about 100% identical to the amino acid sequences set forth in SEQ ID NO: 41, SEQ ID NO: 42, SEQ ID NO: 43, respectively, wherein LCDR1, LCDR2 and LCDR3 have amino acid sequences about 80% to about 100% identical to the amino acid sequences set forth in SEQ ID NO: 44, SEQ ID NO: 45, SEQ ID NO: 46, respectively;
(b) wherein HCDR1, HCDR2 and HCDR3 have amino acid sequences about 80% to about 100% identical to the amino acid sequences set forth in SEQ ID NO: 48, SEQ ID NO: 50, SEQ ID NO: 52, respectively, wherein LCDR1, LCDR2 and LCDR3 have amino acid sequences about 80% to about 100% identical to the amino acid sequences set forth in SEQ ID NO: 55, SEQ ID NO: 57, SEQ ID NO: 59, respectively; or
(c) wherein HCDR1, HCDR2 and HCDR3 have amino acid sequences about 80% to about 100% identical to the amino acid sequences set forth in SEQ ID NO: 62, SEQ ID NO: 64, SEQ ID NO: 66, respectively, wherein LCDR1, LCDR2 and LCDR3 have amino acid sequences about 80% to about 100% identical to the amino acid sequences set forth in SEQ ID NO: 69, SEQ ID NO: 71, SEQ ID NO: 73, respectively.

2. The CAR of claim 1, wherein V_{H} is located at the N-terminus of V_{L}.

3. The CAR of claim 1, wherein V_{H} and V_{L} have amino acid sequences about 80% to about 100% identical to amino acid sequences set forth in (a) SEQ ID NO: 7 and SEQ ID NO: 11, respectively; (b) SEQ ID NO: 9 and SEQ ID NO: 13, respectively; or (c) SEQ ID NO: 33 and SEQ ID NO: 35, respectively.

4. The CAR of claim 1, wherein the anti-CD20 antigen-binding region is a single-chain variable fragment (scFv) that specifically binds CD20.

5. The CAR of claim 1, wherein the CAR further comprises one or more of the following:
(a) a signal peptide,
(b) a hinge region,
(c) a transmembrane domain,
(d) a co-stimulatory region, and
(e) a cytoplasmic signaling domain.

6. The CAR of claim 5, wherein the co-stimulatory region comprises a co-stimulatory region of 4-1BB (CD137), CD28, or combinations thereof.

7. The CAR of claim 5, wherein the cytoplasmic signaling domain comprises a cytoplasmic signaling domain of CD3ζ.

8. The CAR of claim 5, wherein the hinge region comprises a hinge region of CD8, CD28, CD137, IG4, or combinations thereof.

9. The CAR of claim 5, wherein the transmembrane domain comprises a transmembrane domain of CD8, CD28, or combinations thereof.

10. The CAR of claim 1, comprising an amino acid sequence about 80% to about 100% identical to the amino acid sequence set forth in SEQ ID NO: 1, SEQ ID NO: 3, SEQ ID NO: 5, SEQ ID NO: 29, or SEQ ID NO: 31.

11. The CAR of claim 5, wherein the hinge region comprises an amino acid sequence about 80% to about 100% identical to the amino acid sequence set forth in SEQ ID NO: 17, or SEQ ID NO: 19.

12. The CAR of claim 5, wherein the transmembrane domain comprises an amino acid sequence about 80% to about 100% identical to the amino acid sequence set forth in SEQ ID NO: 21.

13. The CAR of claim 5, wherein the cytoplasmic signaling domain comprises an amino acid sequence about 80% to about 100% identical to the amino acid sequence set forth in SEQ ID NO: 25.

14. The CAR of claim 5, wherein the co-stimulatory region comprises an amino acid sequence about 80% to about 100% identical to the amino acid sequence set forth in SEQ ID NO: 23, or SEQ ID NO: 39.

15. An immune cell expressing the CAR of claim 1.

16. The immune cell of claim 15, wherein the immune cell is a T cell or a natural killer (NK) cell.

17. A pharmaceutical composition comprising the immune cell of claim 15.

18-39. (canceled)
